SEQUENCE LISTING (6 sequences)

SEQ ID NO: 1 - DNA, 26 bp, Artificial sequence
  Synthetic primer/probe
tgggaaggggtcagctctatgtggta

SEQ ID NO: 2 - DNA, 32 bp, Artificial sequence
  Synthetic primer/probe
ccttccccttccttcctcttttcctccgttat

SEQ ID NO: 3 - DNA, 25 bp, Artificial sequence
  Synthetic primer/probe
gtcatttcacagctggcaagaggta

SEQ ID NO: 4 - DNA, 27 bp, Artificial sequence
  Synthetic primer/probe
caggaagtggaggtgatagggacaaca

SEQ ID NO: 5 - protein, 517 aa, Homo sapiens
MKGTKDLTSQQKESNVKTFCSKNILAILGFSSIIAVIALLAVGLTQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKTRWFSIVPYETNNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHSTYVFLMVLFSLVLFTVAIIGLLIFHKPSYFWKDMY

SEQ ID NO: 6 - protein, 574 aa, Homo sapiens [sequence omitted]

(19) United States
(12) Patent Application Publication
(10) Pub. No.: US 2019/0160029 A1
(43) Pub. Date: May 30, 2019

(54) COMPOSITIONS AND METHODS FOR THE TREATMENT OF PULMONARY ARTERIAL HYPERTENSION

(71) Applicant: The Regents of the University of Michigan, Ann Arbor, MI (US)

(72) Inventors: David Pinsky, Ann Arbor, MI (US); Scott H. Visovatti, Ann Arbor, MI (US)

(21) Appl. No.: 16/098,330

(22) PCT Filed: May 2, 2017
(86) PCT No.: PCT/US2017/030592

§ 371 Date: Nov. 1, 2018

Related U.S. Application Data
(60) Provisional application No. 62/330,520, filed on May 2, 2016.

(51) Int. Cl. A61K 31/185; A61K 38/46; A61K 31/7076; A61K 45/06; A61P 9/12

(52) U.S. Cl. CPC A61K 31/185; A61K 38/46; A61P 9/12; A61K 45/06; A61K 31/7076

(57) ABSTRACT

Provided herein are compositions and methods for the treatment of pulmonary arterial hypertension. In particular, compositions and methods are provided that address purinergic dysregulation, the causes thereof, and/or the effect of downstream targets.

### CROSS-REFERENCE TO RELATED APPLICATIONS

The present application claims the priority benefit of U.S. Provisional Patent Application 62/330,520, filed May 2, 2016, which is incorporated by reference in its entirety.

### STATEMENT REGARDING FEDERAL FUNDING

This invention was made with government support under grant numbers HL127151, NS087147, and HL119623 awarded by the National Institutes of Health. The government has certain rights in the invention.

### FIELD

Provided herein are compositions and methods for the treatment of pulmonary arterial hypertension. In particular, compositions and methods are provided that address purinergic dysregulation, the causes thereof, and/or the effect of downstream targets.

### BACKGROUND

Pulmonary arterial hypertension (PAH) is a progressive disorder characterized by pulmonary arterial vasoconstriction, vascular remodeling, and smooth muscle cell proliferation (ref 33; incorporated by reference in its entirety). The resultant increase in pulmonary vascular resistance (PVR) leads to right ventricular afterload, hypertrophy and, ultimately, death due to right heart failure. Though the triggers of PAH are poorly understood, imbalances involving the prostacyclin, nitric oxide and endothelin-1 pathways have been implicated, and therapies targeting these pathways are currently used to treat the disease (refs. 33, 50; incorporated by reference in their entireties). Common features of successful targeting of the implicated pathways include vasodilation and inhibition of vascular remodeling. However, the prognosis for PAH remains poor despite treatment targeting the known pathways, with a 15% one-year mortality despite modern treatment options (ref 53; incorporated by reference in its entirety). Thus, the development of therapies targeting novel pathways that contribute to the pathobiology of PAH is of great importance.

The purinergic nucleotides adenosine triphosphate (ATP), adenosine diphosphate (ADP), adenosine monophosphate (AMP), and the nucleoside adenosine are extracellular signaling molecules (ref 6; incorporated by reference in its entirety) that can signal downstream effector targets to modulate endothelial and smooth muscle cell growth (ref 32; incorporated by reference in its entirety), apoptosis (ref 13; incorporated by reference in its entirety), coagulation (ref 31; incorporated by reference in its entirety), vascular tone (refs. 7, 8; incorporated by reference in their entireties) and inflammation (ref 16; incorporated by reference in its entirety). These ligands interact with a variety of cognate P1 (adenosine) and P2 (ATP and ADP) receptors to produce effects that may be complimentary or antagonistic to one another, depending upon tissue-specific receptor sub-types and concentrations (ref 7; incorporated by reference in its entirety).

### SUMMARY

Provided herein are compositions and methods for the treatment of pulmonary arterial hypertension. In particular, compositions and methods are provided that address purinergic dysregulation, the causes thereof, and/or the effect of downstream targets.

In some embodiments, provided herein are methods of treating pulmonary arterial hypertension (PAH) in a subject, comprising administering to the subject an agent that degrades extraellular ATP. In some embodiments, the agent dephosphorylates ATP to form ADP, AMP, and/or adenosine. In other embodiments, the agent dephosphorylates ADP or AMP. In some embodiments, the agent is an enzyme. In some embodiments, the enzyme is selected from the group consisting of an ectonucleotidase, an ectonucleotide pyrophosphatase/phosphodiesterase, and an alkaline phosphatase. In some embodiments, the enzyme is ectonucleoside triphosphate diphosphohydrolase-1 (CD39) or an active variant or fragment thereof. In some embodiments, a polypeptide comprising the enzyme is directly administered to the subject (e.g., in a pharmaceutical composition). In other embodiments, a catalytic antibody with enzyme-like properties is administered. In other embodiments, metal ions serve as the catalytic agents. In some embodiments, a polynucleotide encoding the enzyme (e.g., within a suitable vector) is administered to the subject, under conditions such that the enzyme is expressed within cells of the subject and then released into the extracellular environment.

In some embodiments, provided herein are methods of treating pulmonary arterial hypertension (PAH) in a subject, comprising administering to the subject an adenosine analogue. In some embodiments, the adenosine analogue is an adenosine receptor agonist. In some embodiments, the adenosine analogue is an A2A receptor agonist. In some embodiments, the A2A receptor agonist is selected from the group consisting of: ATL-146e, YT-146 (2-(1-octynyl)adenosine), CGS-21680, DPMA (N6-(2-(3,5-dimethoxyphenyl)-2-(2-methylphenyl)ethyl)adenosine), Regadenoson, UK-432,097, Limonene, and NECA (5′-(N-Ethylcarboxamido)adenosine). In some embodiments, the adenosine analogue is an A2B receptor agonist. In some embodiments, the A2B receptor agonist is selected from the group consisting of: BAY 60-6583, NECA (N-ethylcarboxamidoadenosine), (S)-PHPNECA, LUF-5835, and LUF-5845. In some embodiments, the adenosine analogue is an A3 receptor agonist. In some embodiments, the A3 receptor agonist is selected from the group consisting of: 2-(1-Hexynyl)-N-methyladenosine, CF-101 (IB-MECA), CF-102, 2-Cl-IB-MECA, CP-532,903, Inosine, LUF-6000, and MRS-3558. In some embodiments, the adenosine analogue is stable in an extracellular physiological environment.

In some embodiments, provided herein are methods of treating pulmonary arterial hypertension (PAH) in a subject, comprising administering to the subject purinergic receptor antagonist. In some embodiments, the purinergic receptor is selected from the group consisting of P2X1, P2X2, P2X3, P2X4, P2X5, P2Y1, P2Y2, P2Y4, P2Y6, P2Y12, ADORA1, ADORA2A, ADORA2B, and ADORA3. In some embodiments, methods comprise administering a P2X1 antagonist. In some embodiments, the P2X1 antagonist is selected from the list consisting of: NF449, NF279, TNP-ATP triethylammonium salt, Suramin, PPADS (pyridoxalphosphate-6-azophenyl-2′,4′-disulfonic acid), NF 023, Adenosine 2′,5′-diphosphate, PPNDS (Pyridoxal-5′-phosphate-6-(2′-naphthyl azo-6′-nitro-4′,8′-disulfonate)), Ro 0437626, and MRS 2159. In some embodiments, the purinergic receptor is a small molecule. In some embodiments, the purinergic receptor is a peptide. In some embodiments, the purinergic receptor is an antibody or antibody fragment.

In some embodiments, provided herein are methods of treating pulmonary arterial hypertension (PAH) in a subject, comprising administering to the subject an inhibitor of ATP synthesis and/or ATP-release from cells into the extracellular environment.

In some embodiments, agents, therapeutics, polypeptide, polynucleotides, small molecules, peptide, antibodies, antibody fragments, pharmaceutical compositions, etc. are administered to the subject systemically (e.g., by any suitable route). In some embodiments, agents, therapeutics, polypeptide, polynucleotides, small molecules, peptide, antibodies, antibody fragments, pharmaceutical compositions, etc. are administered to the subject locally to the pulmonary system or vascular system. In some embodiments, local administration is to the superior vena cava, the right atrium, or a pulmonary artery.

In some embodiments, therapeutic compositions and methods described herein comprise co-administration with a treatment for PAH and/or PAH symptoms selected from the group consisting of: diuretics, digoxins, blood thinners, calcium channel blockers, vasoactive substances, prostaglandins/prostanoids, endothelin receptor antagonists, phosphodiesterase type 5 inhibitors, nitric oxide pathway antagonists, and activators of soluble guanylate cyclase. In some embodiments, therapeutic compositions and methods described herein comprise co-administration with a treatment for PAH and/or PAH symptoms selected from the group consisting of: an anticoagulant, an antithrombotic agent, an antiplatelet agent, an anti-inflammatory agent, an anti-proliferative/immunosuppressive agent, a cytostatic drug, an antioxidant.

### BRIEF DESCRIPTION OF THE DRAWINGS

FIGS. 1A-C. Decreased CD39 expression on endothelium of pulmonary arteries and plexiform lesions from patients with IPAH. (a) Representative immunohistochemical images of lung tissue stained for CD31 and CD39 shows a marked decrease in endothelial CD 39 in the IPAH patient (right) compared to control tissue from donors with healthy lungs (left). Images are representative of findings in lung tissue from 3 patients in each group. (b) A representative plexiform lesion from a patient with advanced IPAH shows a characteristic network of CD31-positive, endothelial-lined vascular channels that lack CD39 expression. Lesion is representative of those found throughout the lungs of 3 IPAH patients studied. A quantitative comparison of CD39 endothelial staining in 5 IPAH and 5 donor controls is shown in (c). Expression is unchanged in pulmonary respiratory epithelium. Representative lung tissue sections from two IPAH patients and two control patients without lung disease show similar bronchial epithelial CD39 staining (arrows). Arrowheads identify small, peri-bronchial vessels without (IPAH Patient #2) and with (Control Patient #2) endothelial CD39 staining.

### Scale bar 50 μm.

FIGS. 2A-F. Circulating plasma nucleotide and adenosine concentrations are significantly altered in hypoxic CD39^{-/-} mice. Shown are plasma ATP (a), ADP (b), AMP (c), and adenosine (d) concentrations, and the ATP:AMP and ATP:adenosine ratios (f), as determined by liquid chromatography-mass spectrometry. N=6 per group. Values presented as mean±SEM. *p<0.05, **p<0.005.

FIGS. 3A-D. CD39 gene deletion exacerbates hypoxia-induced pulmonary arterial hypertension. Representative, superimposed waveforms of right ventricular systolic pressures [RVSP (a)] and pulmonary arterial pressures (b) are shown for CD39^{-/-} (blue) and CD39^{+/+} (green) mice exposed to hypoxia. Graphs compare the mean RVSP (c) and mean pulmonary arterial pressures (d) for CD39^{+/+} and CD39^{-/-} mice exposed to normoxia or hypoxia for 4 weeks. N=6 for each group in c and d and values are presented as mean±SEM. *p<0.05, **p<0.005.

FIGS. 4A-D. CD39 deletion exacerbates pulmonary arterial remodeling and right ventricular hypertrophy in mice exposed to hypoxia. (a) Representative immunohistochemical staining of CD39 on small pulmonary arteries (arrows) and airway epithelium (arrowheads) in CD39^{+/+}, but not CD39^{-/-}, mice exposed to both normoxia and hypoxia. 60× magnification. Scale bars are equal to 30 μm. (b) Representative photomicrographs using α-SMA antibody and immunofluorescence labeling shows increased pulmonary arterial remodeling in hypoxic CD39^{-/-} mice (60× magnification). Scale bars are equal to 30 μm. (c) Differences in pulmonary arterial medial thickness were determined in small (<50 μm), medium (51-100 μm) and large (>100 μm) pulmonary vessels labeled with α-SMA antibody. For each vessel size category, the values presented are the mean±SEM using 10 fields from 6 mice from each experimental condition. (d) Comparison of right ventricular hypertrophy using Fulton's Index (ratio of RV:LV+septum weights) in wild-type (CD39^{+/+}) and CD39 knockout (CD39^{-/-}) mice exposed to normoxia and hypoxia). N=6 for each group, values presented as mean±SEM. *p<0.05, **p<0.005.

FIGS. 5A-C. Treatment with apyrase or a P2X1 antagonist mitigates severe pulmonary hypertension. P2X1 receptor mRNA increases 8-fold in CD39^{+/+} and 17-fold in CD39^{-/-} mice exposed to four weeks of hypoxia (a). Representative P2X1 receptor (P2RX1) immunohistochemical staining in CD39^{+/+} and CD39^{-/-} mice exposed to normoxia or hypoxia shows increased vessel P2RX1 expression in hypoxic CD39^{+/+} and, to a greater extent, hypoxic CD39^{-/-} mice (b). The effects of 4 weeks of continuously-administered apyrase (apyr), the P2RX1 antagonist NF279, or saline control on right ventricular systolic pressure (RVSP) in the mouse groups indicated are shown in (c). N=5 for each group.

FIG. 6. Increased pulmonary vascular P2X1 receptor expression in IPAH patients. Representative lung tissue sections from two IPAH patients show increased medial P2X1 staining in plexiform lesions, muscularized vessels, and large pulmonary arteries compared to vessels from control patients without pulmonary disease. Size bars equal 50 microns.

FIG. 7. Exercise plasma concentrations of ADO, AMP, ADP, and ATP for subjects divided into: healthy control group, low-risk SSc group, high-risk without PAH group, high-risk with PAH group.

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise. Thus, for example, reference to “a P2X1 inhibiting agent” is a reference to one or more P2X1 inhibiting agents and equivalents thereof known to those skilled in the art, and so forth.

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, a “conservative” amino acid substitution refers to the substitution of an amino acid in a peptide or polypeptide with another amino acid having similar chemical properties, such as size or charge. For purposes of the present disclosure, each of the following eight groups contains amino acids that are conservative substitutions for one another:
1) Alanine (A) and Glycine (G);
2) Aspartic acid (D) and Glutamic acid (E);
3) Asparagine (N) and Glutamine (Q);
4) Arginine (R) and Lysine (K);
5) Isoleucine (I), Leucine (L), Methionine (M), and Valine (V);
6) Phenylalanine (F), Tyrosine (Y), and Tryptophan (W);
7) Serine (S) and Threonine (T); and
8) Cysteine (C) and Methionine (M).

Naturally occurring residues may be divided into classes based on common side chain properties, for example: polar positive (histidine (H), lysine (K), and arginine (R)); polar negative (aspartic acid (D), glutamic acid (E)); polar neutral (serine (S), threonine (T), asparagine (N), glutamine (Q)); non-polar aliphatic (alanine (A), valine (V), leucine (L), isoleucine (I), methionine (M)); non-polar aromatic (phenylalanine (F), tyrosine (Y), tryptophan (W)); proline and glycine; and cysteine. As used herein, a “semi-conservative” amino acid substitution refers to the substitution of an amino acid in a peptide or polypeptide with another amino acid within the same class.

In some embodiments, unless otherwise specified, a conservative or semi-conservative amino acid substitution may also encompass non-naturally occurring amino acid residues that have similar chemical properties to the natural residue. These non-natural residues are typically incorporated by chemical peptide synthesis rather than by synthesis in biological systems. These include, but are not limited to, peptidomimetics and other reversed or inverted forms of amino acid moieties. Embodiments herein may, in some embodiments, be limited to natural amino acids, non-natural amino acids, and/or amino acid analogs.

“Non-conservative substitutions” involve the exchange of a member of one class for a member from another class.

As used herein, the term “sequence identity” refers to the degree to which two polymer sequences (e.g., peptide, polypeptide, nucleic acid, etc.) have the same sequential composition of monomer subunits. The term “sequence similarity” refers to the degree with which two polymer sequences (e.g., peptide, polypeptide, nucleic acid, etc.) differ only by conservative and/or semi-conservative amino acid substitutions. The “percent sequence identity” (or “percent sequence similarity”) is calculated by: (1) comparing two optimally aligned sequences over a window of comparison (e.g., the length of the longer sequence, the length of the shorter sequence, a specified window, etc.), (2) determining the number of positions containing identical (or similar) monomers (e.g., same amino acids occurs in both sequences, similar amino acid occurs in both sequences) to yield the number of matched positions, (3) dividing the number of matched positions by the total number of positions in the comparison window (e.g., the length of the longer sequence, the length of the shorter sequence, a specified window), and (4) multiplying the result by 100 to yield the percent sequence identity or percent sequence similarity. For example, if peptides A and B are both 20 amino acids in length and have identical amino acids at all but 1 position, then peptide A and peptide B have 95% sequence identity. If the amino acids at the non-identical position shared the same biophysical characteristics (e.g., both were acidic), then peptide A and peptide B would have 100% sequence similarity. As another example, if peptide C is 20 amino acids in length and peptide D is 15 amino acids in length, and 14 out of 15 amino acids in peptide D are identical to those of a portion of peptide C, then peptides C and D have 70% sequence identity, but peptide D has 93.3% sequence identity to an optimal comparison window of peptide C. For the purpose of calculating “percent sequence identity” (or “percent sequence similarity”) herein, any gaps in aligned sequences are treated as mismatches at that position.

Any polypeptides described herein as having a particular percent sequence identity or similarity (e.g., at least 70%) with a reference sequence ID number, may also be expressed as having a maximum number of substitutions with respect to that reference sequence. For example, a sequence having at least 90% sequence identity with SEQ ID NO:Z, which is 101 amino acids in length, may have up to 10 substitutions relative to SEQ ID NO:Z, and may therefore also be expressed as having 10 or fewer substitutions relative to SEQ ID NO:Z.

As used herein, the term “subject” broadly refers to any animal, including but not limited to, human and non-human animals (e.g., dogs, cats, cows, horses, sheep, poultry, fish, crustaceans, etc.). As used herein, the term “patient” typically refers to a subject that is being treated for a disease or condition.

As used herein, the term “effective amount” refers to the amount of a composition sufficient to effect beneficial or desired results. An effective amount can be administered in one or more administrations, applications or dosages and is not intended to be limited to a particular formulation or administration route.

As used herein, the term “endogenous,” when used in reference to protein or nucleic acid sequences, refers to a sequence that is native to the subject or species with which it is being employed.

As used herein, the term “exogenous,” when used in reference to protein or nucleic acid sequences, refers to a sequence that is not native to the subject or species with which it is being employed.

As used herein, the terms “administration” and “administering” refer to the act of giving a drug, prodrug, or other agent, or therapeutic treatment to a subject or in vivo, in vitro, or ex vivo cells, tissues, and organs. Exemplary routes of administration to the human body can be through space under the arachnoid membrane of the brain or spinal cord (intrathecal), the eyes (ophthalmic), mouth (oral), skin (topical or transdermal), nose (nasal), lungs (inhalant), oral mucosa (buccal), ear, rectal, vaginal, by injection (e.g., intravenously, subcutaneously, intratumorally, intraperitoneally, etc.) and the like.

As used herein, the terms “co-administration” and “co-administering” refer to the administration of at least two agent(s) (e.g., a P2X1 inhibitor and stable adenosine analogue) or therapies to a subject. In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s), and/or when co-administration of two or more agents results in sensitization of a subject to beneficial effects of one of the agents via co-administration of the other agent.

As used herein, the term “pharmaceutical composition” refers to the combination of an active agent with a carrier, inert or active, making the composition especially suitable for diagnostic or therapeutic use in vitro, in vivo or ex vivo.

The terms “pharmaceutically acceptable” or “pharmacologically acceptable,” as used herein, refer to compositions that do not substantially produce adverse reactions, e.g., toxic, allergic, or immunological reactions, when administered to a subject.

As used herein, the term “pharmaceutically acceptable carrier” refers to any of the standard pharmaceutical carriers including, but not limited to, phosphate buffered saline solution, water, emulsions (e.g., such as an oil/water or water/oil emulsions), and various types of wetting agents, any and all solvents, dispersion media, coatings, sodium lauryl sulfate, isotonic and absorption delaying agents, disintigrants (e.g., potato starch or sodium starch glycolate), and the like. The compositions also can include stabilizers and preservatives. For examples of carriers, stabilizers and adjuvants, see, e.g., Martin, Remington's Pharmaceutical Sciences, 15th Ed., Mack Publ. Co., Easton, Pa. (1975), incorporated herein by reference in its entirety.

As used herein, the term “antibody” refers to a whole antibody molecule or a fragment thereof (e.g., Fab, Fab′, F(ab′)2, Fv, scFv, Fd, diabodies, and other antibody fragments that retain at least a portion of the variable region of an intact antibody. See, e.g., Hudson et al. (2003) Nat. Med. 9:129-134; herein incorporated by reference in its entirety); it may be a polyclonal or monoclonal antibody, chimeric, a humanized, etc.

As used herein, when an antibody or other entity “specifically recognizes” or “specifically binds” an antigen or epitope, it preferentially recognizes the antigen in a complex mixture of proteins and/or macromolecules, and binds the antigen or epitope with affinity which is substantially higher than to other entities not displaying the antigen or epitope. In this regard, “affinity which is substantially higher” means affinity that is high enough to enable detection of an antigen or epitope which is distinguished from entities using a desired assay or measurement apparatus. Typically, it means binding affinity having a binding constant (Kₐ) of at least 10⁷ M⁻¹ (e.g., >10⁷ M⁻¹, >10⁸M⁻¹, >10⁹M⁻¹, >10¹⁰ M⁻¹, >10¹¹ M⁻¹, >10¹² M⁻¹, >10¹³ M⁻¹, etc.). In certain such embodiments, an antibody is capable of binding different antigens so long as the different antigens comprise that particular epitope. In certain instances, for example, homologous proteins from different species may comprise the same epitope.

### DETAILED DESCRIPTION

Provided herein are compositions and methods for the treatment of pulmonary arterial hypertension. In particular, compositions and methods are provided that address purinergic dysregulation, the causes thereof, and/or the effect of downstream targets.

Intravascular nucleotide concentrations are regulated primarily by the ectonucleotidase CD39 (ENTPD1) and CD73 (5′-nucleotidase) (refs. 26, 45, 58; incorporated by reference in their entireties). CD39 phosphohydrolizes ATP and ADP to AMP, which is further dephosphorylated to adenosine by CD73. Thus, these ecto-enzymes play a critical role in maintaining extracellular nucleotide and adenosine homeostasis.

Experiments were conducted during development of embodiments herein to determine if an imbalance in extracellular nucleotide and adenosine ratios caused by altered CD39 expression plays a role in the pathobiology of PAH. To further investigate the role of CD39 and purinergic signaling in PAH, the murine chronic hypoxia model, which is commonly used to investigate pathways that contribute to PAH (refs. 9, 10, 20, 52; incorporated by reference in their entireties). Deletion of the CD39 gene in the setting of chronic hypoxia resulted in significantly altered concentrations of plasma nucleotides and adenosine, significant up-regulation of the lung P2X1 receptor, and a consistent and unexpectedly severe pulmonary hypertension phenotype. Furthermore, reconstitution of CD39 using a soluble apyrase mitigates the development of PAH, while antagonism of the P2X1 receptor prevents the development of PAH altogether.

Despite the fact that nucleotides and adenosine help regulate vascular tone through purinergic signaling pathways, little has been understood regarding their contributions to the pathobiology of pulmonary arterial hypertension, a condition characterized by elevated pulmonary vascular resistance and remodeling. Even less has been known about any roles that alterations in CD39 (ENTPD1) and/or CD73 (ecto-5′-nucleotidase, ecto-5′-NT) the ectonucleotidase responsible for the conversion of the nucleotides ATP and ADP to AMP, and the nucleotidase responsible for the conversion of AMP to adenosine, respectively, may play in pulmonary arterial hypertension. Experiments conducted during development of embodiments herein identified decreased CD39 expression on the pulmonary endothelium of patients with idiopathic pulmonary arterial hypertension. Through examining the effects of CD39 gene deletion in mice exposed to normoxia or normobaric hypoxia (10% oxygen), it was found that hypoxic CD39^{-/-} mice have a markedly elevated ATP-to-adenosine ratio, higher pulmonary arterial pressures, more right ventricular hypertrophy, more arterial medial hypertrophy, and a pro-thrombotic phenotype (e.g., a PAH-like state). In addition, hypoxic CD39^{-/-} mice exhibited a marked increase in lung P2X1 receptors. Systemic reconstitution of ATPase and ADPase enzymatic activities through continuous administration of apyrase decreased pulmonary arterial pressures in hypoxic CD39^{-/-} mice to levels found in hypoxic CD39^{+/+} controls. Treatment with NF279, a potent and specific P2X1 receptor antagonist, lowered pulmonary arterial pressures even further. The experiments conducted during development of embodiments herein implicate decreased CD39 and resultant alterations in circulating purinergic signaling ligands and cognate receptors in the pathobiology of pulmonary arterial hypertension. Reconstitution and receptor blocking experiments indicate that phosphohydrolysis of purinergic nucleotide tri- and di-phosphates, or blocking of the P2X1 provides treatment for pulmonary arterial hypertension (PAH).

Experiments conducted during development of embodiments herein demonstrate that deletion of CD39 in vivo results in an increase in the plasma ATP-to-adenosine ratio, high pulmonary arterial pressures, significant pulmonary artery remodeling and right ventricular hypertrophy in a murine hypoxia model. Marked increases in both circulating intravascular ATP and cognate lung P2X1 receptors identify a previously unidentified mechanism by which altered purinergic signaling contributes to the pathobiology of pulmonary arterial hypertension. Furthermore, reconstitution of CD39^{-/-} mice using soluble apyrase with ATPase and ADPase activities lowered pulmonary arterial pressures to levels seen in hypoxic wild-type mice, and treatment with a potent and specific P2X1 receptor prevented the development of pulmonary hypertension altogether. These findings implicate altered CD39 activity, a shift towards a high ATP and low adenosine intravascular environment, and an increase in the P2X1 receptor in the development of PH.

In vivo experiments conducted during development of embodiments herein demonstrate impressive alterations in a full panel of circulating plasma nucleotide and adenosine concentrations as a direct result of targeted deleting the CD39 gene. Methods included the use of a LC-MS platform to analyze plasma drawn directly into a “stop solution” formulated to minimize the inter-conversion of ATP, ADP, AMP and adenosine. The role of intravascular nucleotides and adenosine as extracellular signaling molecules is well-established (ref 7; incorporated by reference in its entirety). ATP is released in both lytic and non-lytic manners from the major blood vessel wall cell types, including endothelial cells (ref 4; incorporated by reference in its entirety), vascular smooth muscle cells (ref 27; incorporated by reference in its entirety), perivascular sympathetic nerves (ref 29; incorporated by reference in its entirety), and erythrocytes (ref 3; incorporated by reference in its entirety). The finding of a hypoxia-induced increase in circulating plasma ATP in CD39^{+/+} wild-type mice is consistent with prior studies in which hypoxia has been shown to increase luminal ATP concentrations with contributions from circulating erythrocytes (ref 3; incorporated by reference in its entirety) and endothelium (ref 4; incorporated by reference in its entirety). However, the finding of an extreme elevation of plasma ATP (and a lesser though significant increase in ADP) in hypoxic CD39^{-/-} mice was unexpected. These findings indicate that the lack of the ectoenzyme CD39 results in a large buildup of upstream substrates (ATP and ADP) and dearth of downstream products (adenosine and a trend towards decreased AMP).

While CD39 is the major extracellular ectonucleotidase (ref 41; incorporated by reference in its entirety), other enzymes such as the ectonucleotide pyrophosphatase/phosphodiesterase (E-NPP) family and alkaline phosphatases are capable of hydrolyzing ATP, while adenylate kinase, nucleoside diphosphate kinase and ATP synthase can regenerate ATP (ref 58; incorporated by reference in its entirety). In addition, the concentration of intravascular nucleotides is altered by cell lysis, release channels, transporters and exocytosis (ref 58; incorporated by reference in its entirety). It is contemplated that these other mechanisms explain the finding of increased circulating ATP and ADP in hypoxic, but not normoxic, CD39^{-/-} mice (FIGS. 2a and 2b). This multi-faceted system is capable of maintaining nucleotide homeostasis in normoxic conditions, despite a lack of CD39, but hypoxia overwhelms these mechanisms resulting in the high ATP and ADP concentrations noted in the experiments conducted during development of embodiments herein.

Based upon the finding of an ATP-rich and adenosine-poor intravascular environment in hypoxic CD39^{-/-} mice, experiments conducted during development of embodiments herein to demonstrate that these changes perturb purinergic signaling homeostasis, leading to increased pulmonary arterial pressures. The intravascular nucleotides and adenosine are important signaling molecules that regulate the cardiovascular system by acting upon P1 (adenosine) and P2 receptors (ATP and ADP).

The continuous infusion of soluble apyrase, which cleaves inorganic phosphate from ATP and ADP, provided partial protection against the development of pulmonary hypertension in CD39^{-/-} mice exposed to four weeks of hypoxia. the finding of a protective role for a substance with both ATPase and ADPase activity (e.g., CD39) against the development of PAH sheds light on the increased functional CD39 on circulating plasma microparticles in patients with PAH (ref 56; incorporated by reference in its entirety). Microparticle-based CD39 in humans is increased as a compensatory response to mitigate PAH. The experiments conducted during development of embodiments herein findings are congruent with previous studies that have shown CD39 to be protective in hypoxic environments contributing to other disease states (ref 15, 21; incorporated by reference in their entireties). Continuous administration of apyrase did not completely protect CD39^{+/+} mice from developing pulmonary hypertension (FIG. 5c). This finding indicates that decreasing the build-up of ATP and ADP alone was not sufficient to completely prevent pulmonary hypertension, and indicated the combined contribution CD39 deletion and hypoxia on purinergic receptors.

Purinergic receptor transcriptomic profiling of lungs from CD39^{+/+} and CD39^{-/-} mice identified significant up-regulation of three receptors in response to hypoxia. The most significant change was in the P2X1 receptor, which showed a near nine-fold increase in hypoxic wild-type mice and a seventeen-fold increase in hypoxic CD39^{-/-} mice. These marked elevations provide mechanistic insight into the role of purinergic signaling in the development of pulmonary hypertension. The P2X1 receptor is expressed at high levels in vascular smooth muscle and platelets, and to lesser degrees in the heart and inflammatory cells (ref 16; incorporated by reference in its entirety). P2X1-receptor-mediated vasoconstriction plays a role in the regulation of afferent renal arterioles (ref 25; incorporated by reference in its entirety), mesenteric arteries, vas deferens (ref 37; incorporated by reference in its entirety) and urinary bladder (ref 55; incorporated by reference in its entirety). P2X1 receptor mRNA has been identified in the smooth muscle of adult rat pulmonary arteries (ref 39; incorporated by reference in its entirety), and it has been suggested that the P2X1 receptor likely mediated a small vasoconstrictive response in isolated adult porcine pulmonary arteries in response to the non-hydrolyzable ATP analogue α,β-meATP (ref 34; incorporated by reference in its entirety). The experiments conducted during development of embodiments herein identified a role for P2X1 receptor and the ATP ligand in the pathogenesis of pulmonary hypertension (e.g., in particular, by an in vivo approach). The potent and selective P2X1 antagonist NF279 (ref 11, 43; incorporated by reference in its entirety) protected both CD39^{+/+} and CD39^{-/-} mice from the development of hypoxic pulmonary hypertension. This finding emphasizes the importance of the P2X1 receptor in the development of hypoxic pulmonary hypertension and demonstrates a disease paradigm in CD39 deletion results in up-regulation of both the P2X1 receptor and its cognate ligand (ATP).

In addition to a marked up-regulation of the P2X1 receptor, profiling also identified a significant increase in the pulmonary A2A receptor (Table 4) and a more modest increase in the pulmonary P2Y2 receptor (Table 3) in response to hypoxia. In both cases, the mRNA was highest in the lungs of CD39^{-/-} mice. Deletion of the A2A receptor resulted in pulmonary hypertension in an experimental model (ref 57; incorporated by reference in its entirety). Thus, it is contemplated that the increase in the A2A receptor in experiments conducted during development of embodiments herein represents a compensatory mechanism. P2Y2 is plays a role in experimental models of systemic hypertension (ref 30; incorporated by reference in its entirety), but its role in the pathobiology of pulmonary hypertension has not been elucidated. Experiments conducted during development of embodiments herein indicate that adenosine receptors are upregulated to in an attempt to compensate for the reduced adenosine concentration.

Based upon the findings in the mouse model experiments, a role for dysregulated purinergic signaling in the pathobiology of human PAH was explored using lung sections from patients with idiopathic pulmonary arterial hypertension (IPAH) and age- and gender-matched controls. Immunohistochemical staining of vascular CD39 showed a decrease in IPAH lungs compared to control lungs. These findings are congruent with a recent study that showed attenuated pulmonary arterial endothelial CD39 in patients with IPAH (ref 23; incorporated by reference in its entirety). However, the in vivo model herein expands upon these findings, identifying a mechanism by which decreased CD39 leads to the development of pulmonary hypertension as a result of increases in both the P2X1 receptor and its cognate ATP ligand. This is relevant to human IPAH, as immunohistochemical staining of human IPAH lungs showed increased vascular P2X1 staining, compared to control lungs. Systemic pressures were not affected by CD39 deletion in the mouse, but pulmonary arterial pressures increased markedly in the setting of hypoxia. This specificity is also present in humans with pulmonary arterial hypertension, who do not exhibit associated systemic hypertension even in the setting of severe elevations of the pulmonary arterial pressures. Thus, the model recapitulates this common presentation of the human disease. It is contemplated that these differences are due to differences in purinergic receptor compositions in systemic and pulmonary vessels.

The experiments conducted during development of embodiments herein provide a mechanism by which severe hypoxia-induced PH develops as a result of CD39 ectonucleotidase dysregulation, a resulting shift in intravascular nucleotide and nucleoside concentration towards an ATP/ADP-rich and AMP/adenosine-poor milieu, and a significant up-regulation of pulmonary vascular P2X1 receptor. Reconstitution of ATPase and ADPase activity lessened the degree to which pulmonary arterial pressures increased in this model, while blockade of the P2X1 receptor prevented an increase in pulmonary arterial pressures altogether. The findings of decreased CD39 and increased P2X1 receptor expression in pulmonary vessels from IPAH patients support dysregulated purinergic signaling as a mechanism contributing to this disease. The therapeutic benefits of (i) CD39 delivery, (ii) P2X1 receptor, or other ATP receptor, antagonism, (iii) ATP degradation, (iv) activation of adenosine receptors, and/or (v) increase in the concentration of stable adenosine analogues, in PAH are apparent based on the experiments conducted during development of embodiments herein.

Experiments conducted during development of embodiments herein elucidate a previously unrecognized and not understood mechanism that leads to PAH and the symptoms associated therewith (e.g., increased pulmonary arterial pressures, increased right ventricular hypertrophy, increased arterial medial hypertrophy, pro-thrombotic phenotype, etc.), and present multiple modes for the treatment of PAH and/or relief of the associated symptoms. These treatment modes include: (i) degradation or sequestration of excess extracellular ATP; (ii) inhibition of ATP receptors (e.g., upregulated ATP receptors (e.g., PX21 receptor); (iii) administration of stable adenosine analogues in order to increase the apparent adenosine concentration and/or decrease the extracellular ratio of ATP:adenosine; and (iv) administration of adenosine receptor agonists to restore downstream signaling. Each of these therapeutic modes, as well as multiple strategies (e.g., compositions and methods) for addressing each mode, is addressed below.

### I. Excess ATP

In some embodiments, an underlying cause of PAH and/or all or a portion of the symptoms associated therewith is excess extracellular or plasma ATP and/or ADP concentrations; although embodiments herein are not limited to any particular mechanism of action and an understanding of the mechanism of action is not necessary to practice such embodiments. In some embodiments, treatment of PAH or PAH symptoms comprises degrading (e.g. dephosphorylating) excess plasma ATP to restore appropriate plasma ATP levels and/or appropriate ratios of ATP to other nucleotides or adenosine.

In some embodiments, excess ATP (and/or ADP) is removed and/or healthy ATP (and/or ADP) levels are restored by the enzymatic degradation (e.g. dephosphorylation) of ATP in a subject. In some embodiments, expression of a subject's endogenous enzymes for the dephosphorylation of ATP (and/or ADP) is enhanced. In some embodiments, a polypeptide comprising an ATP (and/or ADP) dephosphorylating enzyme (e.g., CD39) is administered to a subject. In some embodiments, a nucleic acid (e.g., nucleic acid vector) encoding an ATP (and/or ADP) dephosphorylating enzyme (e.g., CD39) is administered to a subject. In some embodiments, the ATP (and/or ADP) dephosphorylating enzyme, or nucleic acid encoding as much, is an active form of an enzyme that is endogenous to the subject. In some embodiments, the ATP (and/or ADP) dephosphorylating enzyme, or nucleic acid encoding as much, is an active enzyme that is not endogenous (e.g., engineered, from another species) to the subject. In some embodiments, an exogenous ATP (and/or ADP) dephosphorylating enzyme is utilized to evade the issues that have led to in the faulty ATP (and/or ADP) dephosphorylation and resulted in PAH and/or PAH-related symptoms. In some embodiments, suitable enzymes include ATPases, apyrases, ectonucleotide pyrophosphatase/phosphodiesterase (E-NPP) enzymes, alkaline phosphatases, etc. In some embodiments, endogenous or exogenous enzymes are selected for activity in an extracellular environment. In some embodiments, modifications are made to endogenous or exogenous enzymes to optimize activity in an extracellular environment.

Embodiments herein are not limited to any particular ATP (and/or ADP) dephosphorylating enzyme. Either as a cause of PAH or as a treatment thereof.

In some embodiments, extracellular ATP levels are reduced by inhibiting the expression (e.g., RNAi or the like) and/or activity (e.g., an inhibitor or the like) of enzymes that produce or result in the production of ATP. Such enzymes include, for example, adenylate kinase, nucleoside diphosphate kinase and ATP synthase. In some embodiments, extracellular ATP levels are reduced by inhibiting ATP-release from cells. In some embodiments, mechanisms of ATP release (e.g., vesicular exocytosis, plasma membrane F₁/F₀-ATP synthase, ATP-binding cassette (ABC) transporters, connexin hemichannels, pannexin channels, etc.) are inhibited to prevent or reduce ATP release. In some embodiments, expression of proteins, receptors, and/or channels responsible for ATP release are inhibited.

In some embodiments, particularly in which a mutation in an endogenous ATP (and/or ADP) dephosphorylating enzyme (e.g., CD39) is a cause of the excess ATP, techniques (e.g., CRISPR/Cas9) are provided to correct the mutation and restore active ATP (and/or ADP) dephosphorylatinon.

In some embodiments, particularly in which a defective endogenous protein the subject is a cause of the excess ATP, techniques are provided to restore ATP (and/or ADP) dephosphorylation activity to the subject. In some embodiments, inhibitors (e.g., small molecule inhibitors, antagonists, antibodies (or antibody fragments) that bind the defective endogenous protein, etc.) of the activity of the defective protein are employed (e.g., administered). In some embodiments, inhibitors of expression (e.g., RNAi, antisense RNA, etc.) of the defective endogenous protein are employed (e.g., administered).

Experiments conducted during development of embodiments herein indicate a role of CD39 in PAH and/or symptoms thereof. In some embodiments, excess extracellular ATP and/or the downstream effectorss thereof (e.g., upregulated and/or over-active purinergic receptors) are treated and/or ameliorated by the reconstitution of CD39 activity and/or CD39-like activity (e.g., by the administration of a CD39 polypeptide (or nucleic acid encoding a CD39 polypeptide) or a polypeptide capable CD39-like ATP (and/or ADP) dephosphorylation. In some embodiments, a CD39 polypeptide administered, expressed, or encoded (by a nucleic acid and/or vector) in methods or compositions herein comprises at least 60% sequence identity (e.g., 60%, 65%, 70%, 75%, 80%, 85%, 90%, 95%, 99%, or ranges therebetween) to all or a portion of a native, wild-type human CD39 (SEQ ID NO: 5):

| | |
|---|---|
| 1 | mkgtkdltsq qkesnvktfc sknilailgf ssiiaviall avgltqnkal penvkygivl |
| 61 | dagsshtsly iykwpaeken dtgvvhqvee crvkgpgisk fvqkvneigi yltdcmerar |
| 121 | eviprsqhqe tpvylgatag mrllrmesee ladrvldvve rslsnypfdf qgariitgqe |
| 181 | egaygwitin yllgkfsqkt rwfsivpyet nnqetfgald lggastqvtf vpqnqtiesp |
| 241 | dnalqfrlyg kdynvythsf lcygkdqalw qklakdiqva sneilrdpcf hpgykkvvnv |
| 301 | sdlyktpctk rfemtlpfqq feiqgignyq qchqsilelf ntsycpysqc afngiflppl |
| 361 | qgdfgafsaf yfvmkflnlt sekvsqekvt emmkkfcaqp weeiktsyag vkekylseyc |
| 421 | fsgtyilsll lqgyhftads wehihfigki qgsdagwtlg ymlnltnmip aeqplstpls |
| 481 | hstyvflmvl fslvlftvai igllifhkps yfwkdmv. |

In some embodiments, a CD39 polypeptide administered, expressed, or encoded (by a nucleic acid and/or vector) in methods or compositions herein is not a naturally-occurring sequence and/or has less than 100% sequence identity to all or a portion of, for example SEQ ID NO: 5.

### II. Increased Purinergic Receptor (e.g., P2X1) Signaling

In some embodiments, an underlying cause of PAH and/or all or a portion of the symptoms associated therewith is upregulation and/or over-activity (e.g., from overly-high levels of ligand) of purinergic receptors (e.g., ATP-specific (e.g., P2X-type (e.g., P2X1), etc.), purine-specific (e.g., P2Y-type, etc.), etc.); although embodiments herein are not limited to any particular mechanism of action and an understanding of the mechanism of action is not necessary to practice such embodiments. In some embodiments, an underlying cause of PAH and/or all or a portion of the symptoms associated therewith is upregulation and/or over-activity of a purinergic receptor, selected from, for example: P2X1, P2X2, P2X3, P2X4, P2X5, P2Y1, P2Y2, P2Y4, P2Y6, P2Y12, ADORA1, ADORA2A, ADORA2B, and ADORA3. In some embodiments, upregulation and/or over-activity of purinergic receptors (e.g., P2X1, etc.) is the result of increased concentrations of extracellular (plasma) ATP. In some embodiments, treatment of PAH or PAH symptoms comprises inhibiting expression of one or more purinergic receptors (e.g., P2X1, etc.). In some embodiments, inhibitors of expression (e.g., RNAi, antisense RNA, etc.) of the one or more purinergic receptors (e.g., P2X1, etc.) are employed (e.g., administered). In some embodiments, inhibitors of activity (e.g., small molecule, peptide, antibody (or antibody fragment), etc.) of the one or more purinergic receptors (e.g., P2X1, etc.) are employed (e.g., administered).

In some embodiments, a cause of PAH and/or all or a portion of the symptoms associated therewith is upregulation and/or over-activity P2X1. In some embodiments, treatment of PAH and/or PAH symptoms comprises inhibiting expression of P2X1 by, for example, siRNA, antisense RNA, gene therapy, Cas9/CRISPR, etc. In some embodiments, treatment of PAH and/or PAH symptoms comprises inhibiting activity of P2X1 by, for example, administering a small-molecule-, peptide-, antibody-, or antibody-fragment-inhibitor of P2X1 activity. Small molecule inhibitors of P2X1 activity are known in the field, and include:

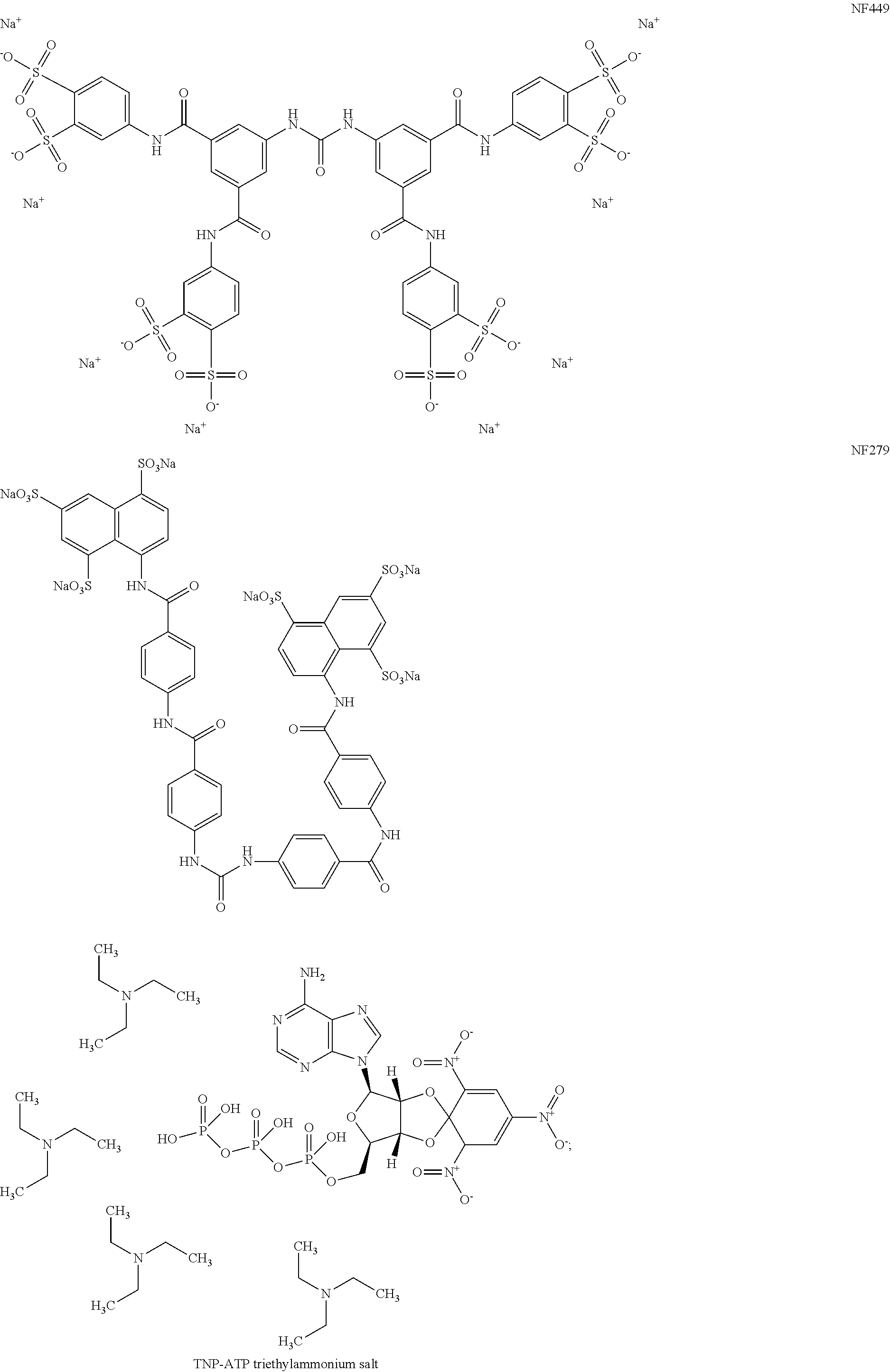

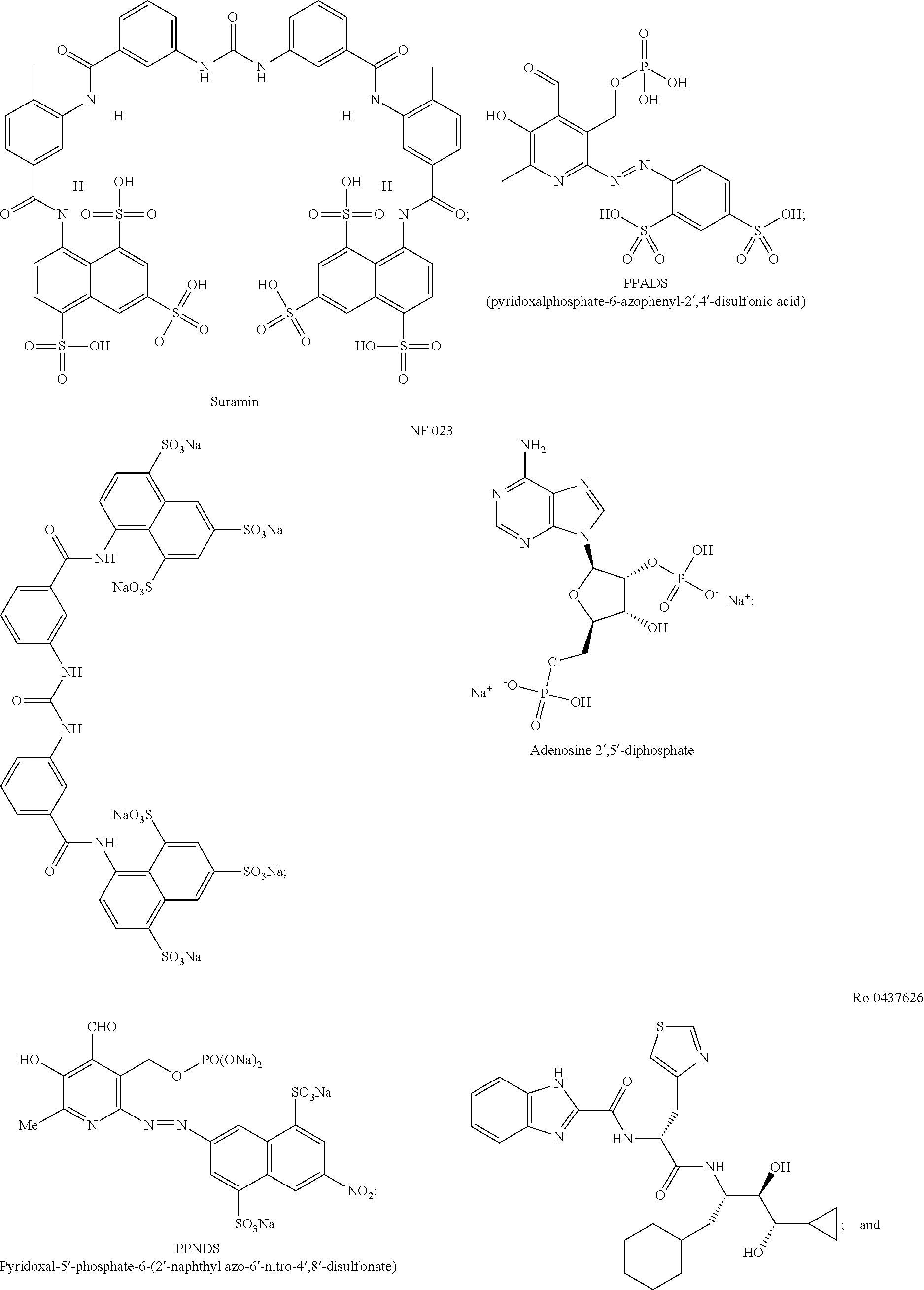

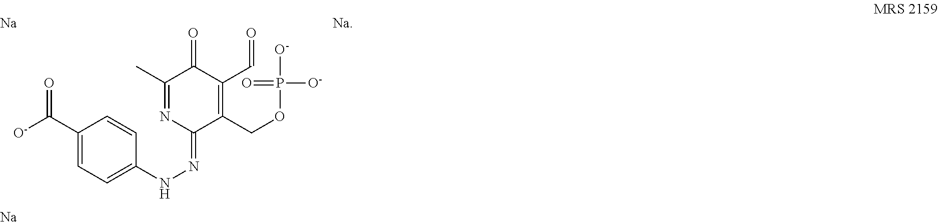

In some embodiments, P2X1 activity is inhibited by the administration of an inhibitory antibody or antibody fragment, which prevents the binding of extracellular ATP to P2X1.

### III. Decreased Extracellular Adenosine Levels

In some embodiments, an underlying cause of PAH and/or all or a portion of the symptoms associated therewith is decreased extracellular and/or plasma concentrations of adenosine (and/or AMP); although embodiments herein are not limited to any particular mechanism of action and an understanding of the mechanism of action is not necessary to practice such embodiments. In some embodiments, treatment of PAH or PAH symptoms comprises administering adenosine or stable analogues of adenosine to restore the apparent plasma adenosine levels and/or apparent ratios of adenosine to ATP or to other nucleotides. In some embodiments, AMP or an AMP analogue is administered to restore the apparent plasma AMP levels and/or provide a substrate for conversion into adenosine. In some embodiments, compositions and methods are provided to convert excess nucleotides (e.g., ATP and/or ADP) into AMP and/or adenosine to restore plasma AMP/adenosine levels. In some embodiments, enzymes are provided (and/or nucleic acids encoding such enzymes) for the conversion of excess ATP and/or ADP into adenosine (and/or AMP).

In some embodiments, adenosine receptor agonists are administered. Such agents include:

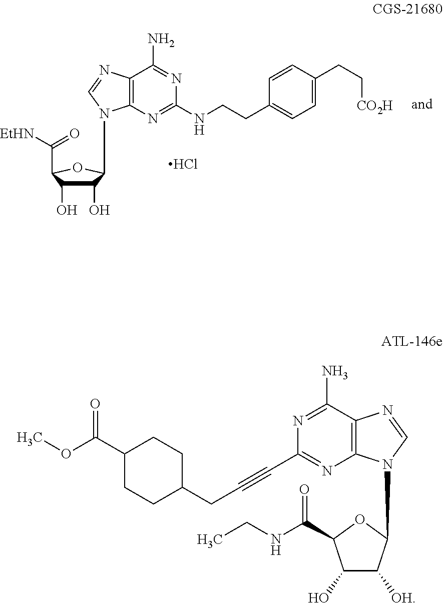

In some embodiments, an adenosine receptor agonist is an A2A receptor agonist, such as, but not limited to: ATL-146e, YT-146 (2-(1-octynyl)adenosine), CGS-21680, DPMA (N6-(2-(3,5-dimethoxyphenyl)-2-(2-methylphenyl)ethyl)adenosine), Regadenoson, UK-432,097, Limonene, and NECA (5′-(N-Ethylcarboxamido)adenosine). In some embodiments, an adenosine receptor agonist is an A2B receptor agonist, such as, but not limited to: BAY 60-6583, NECA (N-ethylcarboxamidoadenosine), (S)-PHPNECA, LUF-5835, and LUF-5845. In some embodiments, an adenosine receptor agonist is an A3 receptor agonist, such as, but not limited to: 2-(1-Hexynyl)-N-methyladenosine, CF-101 (IB-MECA), CF-102, 2-Cl-IB-MECA, CP-532,903, Inosine, LUF-6000, and MRS-3558.

In some embodiments, insufficient extracellular adenosine (and/or AMP) is treated by restoring purinergic regulation pathways in the subject (e.g., pathways that result in the production of adenosine). In some embodiments, expression is enhanced of a subject's endogenous enzymes (e.g., CD39, CD73, etc.) for the production of adenosine (e.g., via dephosphorylating of ATP, ADP, and/or AMP). In some embodiments, nucleotide (e.g., ATP, ADP, AMP) dephosphorylating enzymes (e.g., ectonucleotidases, nucleotidases, etc.) are administered to a subject. In some embodiments, a nucleic acid (e.g., nucleic acid vector) encoding a nucleotide (e.g., ATP, ADP, AMP) dephosphorylating enzyme (e.g., ectonucleotidase, nucleotidase, etc.) is administered to a subject. In some embodiments, the ATP (and/or ADP) dephosphorylating enzyme, or nucleic acid encoding as much, is an active form of an enzyme that is endogenous to the subject. In some embodiments, the ATP (and/or ADP) dephosphorylating enzyme, or nucleic acid encoding as much, is an active enzyme that is not endogenous (e.g., engineered, from another species) to the subject. In some embodiments, an exogenous nucleotide (e.g., ATP, ADP, AMP) dephosphorylating enzyme is utilized to evade the issues that have led to in the faulty adenosine production and resulted in PAH and/or PAH-related symptoms. In some embodiments, the ATP (and/or ADP) dephosphorylating enzyme is an ecto-enzyme, or has been modified to function as one in vivo.

In some embodiments, particularly in which a mutation in an endogenous ATP, ADP, and/or AMP dephosphorylating enzyme (e.g., CD39, CD73, etc.) is a cause of the reduced extracellular adenosine, techniques (e.g., CRISPR/Cas9) are provided to correct the mutation and restore enzyme activity and normal adenosine levels.

In some embodiments, particularly in which a defective endogenous protein the subject is a cause of the reduced adenosine, techniques are provided to restore enzymatic production of adenosine to the subject. In some embodiments, inhibitors (e.g., small molecule inhibitors, antagonists, antibodies (or antibody fragments) that bind the defective endogenous protein, etc.) of the activity of the defective protein are employed (e.g., administered). In some embodiments, inhibitors of expression (e.g., RNAi, antisense RNA, etc.) of the defective endogenous protein are employed (e.g., administered).

Experiments conducted during development of embodiments herein have demonstrated a role of CD39 in PAH and/or symptoms thereof. In some embodiments, it is contemplated that CD73 has a role in PAH and/or symptoms thereof. In some embodiments, reduced extracellular adenosine and/or the downstream effects thereof (e.g., as effected through adenosine receptors) are treated and/or ameliorated by the reconstitution of combined CD39/CD73 activity and/or CD39/CD73-like activity (e.g., by the administration of a CD39 and/or CD73 polypeptide (or nucleic acid encoding a CD39 and/or CD73 polypeptide) or a polypeptide capable CD39/CD73-like conversion of tri-, di-, or mono-phosphorylated adenosine to unphosphorylated adenosine. In some embodiments, a CD39 polypeptide administered, expressed, or encoded (by a nucleic acid and/or vector) in methods or compositions herein comprises at least 60% sequence identity (e.g., 60%, 65%, 70%, 75%, 80%, 85%, 90%, 95%, 99%, or ranges therebetween) to all or a portion of a native, wild-type human CD39 (SEQ ID NO: 5). In some embodiments, a CD39 polypeptide administered, expressed, or encoded (by a nucleic acid and/or vector) in methods or compositions herein is not a naturally-occurring sequence and/or has less than 100% sequence identity to all or a portion of, for example SEQ ID NO: 5. In some embodiments, a CD73 polypeptide administered, expressed, or encoded (by a nucleic acid and/or vector) in methods or compositions herein comprises at least 60% sequence identity (e.g., 60%, 65%, 70%, 75%, 80%, 85%, 90%, 95%, 99%, or ranges therebetween) to all or a portion of a native, wild-type human CD73 (SEQ ID NO: 6):

| | |
|---|---|
| 1 | mcpraarapa tlllalgavl wpaagawelt ilhtndvhsr leqtsedssk cvnasrcmgg |
| 61 | varlftkvqq irraepnvll ldagdqyqgt iwftvykgae vahfmnalry damalgnhef |
| 121 | dngvegliep llkeakfpil sanikakgpl asqisglylp ykvlpvgdev vgivgytske |
| 181 | tpflsnpgtn lvfedeital qpevdklktl nvnkiialgh sgfemdklia qkvrgvdvvv |
| 241 | gghsntflyt gnppskevpa gkypfivtsd dgrkvpvvqa yafgkylgyl kiefdergnv |
| 301 | isshgnpill nssipedpsi kadinkwrik ldnystqelg ktivyldgss qscrfrecnm |
| 361 | gnlicdamin nnlrhadetf wnhvsmciln gggirspide rnngtitwen laavlpfggt |
| 421 | fdlvqlkgst lkkafehsvh rygqstgefl qvggihvvyd lsrkpgdrvv kldvlctkcr |
| 481 | vpsydplkmd evykvilpnf langgdgfqm ikdellrhds gdqdinvvst yiskmkviyp |
| 541 | avegrikfst gshchgsfsl iflslwavif vlyq |

In some embodiments, a CD73 polypeptide administered, expressed, or encoded (by a nucleic acid and/or vector) in methods or compositions herein is not a naturally-occurring sequence and/or has less than 100% sequence identity to all or a portion of, for example SEQ ID NO: 6.

### IV. Therapies/Techniques

### A. Therapeutic Polynucleotides

As described above, experiments conducted during development of embodiments herein demonstrate that aberrant/dysregulated purinergic signaling and/or pathways (e.g., resulting in increased extracellular ATP, decreased extracellular adenosine, upregulated ATP receptors (e.g., P2X1), etc.)) results in PAH, PAH symptoms, and/or a PAH-like state. In some embodiments, methods and compositions are provided for restoring/increasing/supplementing enzymatic activity to restore normal purinergic pathways and bring plasma purine (e.g., adenosine and/or ATP) levels into a healthy range. In some embodiments, enzymatic activity is restored/supplemented by expressing proteins capable of purine metabolism (e.g., catabolizing ATP, anabolizing adenosine). In some embodiments, polynucleotides are administered (e.g., by suitable techniques described herein), that when expressed within a subject, produce enzymes capable of restoring proper ATP/adenosine levels (e.g., ATP/ADP/AMP dephosphorylating enzymes (e.g., CD39, cd73, etc.)). In some embodiments, polynucleotides are administered that express endogenous puurogenic pathway enzymes (e.g., naturally-occurring enzymes, wild-type human enzymes, etc.). In some embodiments, polynucleotides are administered that express exogenous enzymes capable of reducing extracellular ATP levels and/or increasing adenosine levels. In particular embodiments, polynucleotides encoding polypeptides having at least 60% (e.g., 60%, 65%, 70%, 75%, 80%, 85%, 90%, 95%, 100%, or ranges therebetween) sequence identity or similarity with SEQ ID NO: 5 or SEQ ID NO: 6 are administered. In other embodiments, polynucleotides encoding exogenous polypeptides capable of restoring extracellular ATP and/or adenosine levels (e.g., via degradation and/or dephosphorylation of exrtracellular ATP, via dephosphorylation of ATP, ADP, and/or AMP to adenosine, etc.) are administered. In some embodiments, therapeutically effective vectors comprising such polynucleotides are administered. Accordingly, provided herein are nucleic acids (e.g., comprising genes) encoding endogenous and exogenous purinergic pathway enzymes, and methods of administering such nucleic acids to a subject suffering from PAH.

Polynucleotides may be employed for producing polypeptides by recombinant techniques. Thus, for example, the polynucleotide may be included in any one of a variety of expression vectors for expressing a polypeptide. In some embodiments, vectors include, but are not limited to, chromosomal, nonchromosomal and synthetic DNA sequences (e.g., derivatives of SV40, bacterial plasmids, phage DNA; baculovirus, yeast plasmids, vectors derived from combinations of plasmids and phage DNA, and viral DNA such as vaccinia, adenovirus, fowl pox virus, and pseudorabies). It is contemplated that any vector may be used as long as it is replicable and viable in the host (e.g., subject suffering from PAH). Large numbers of suitable vectors are known to those of skill in the art, and are commercially available. In some embodiments, mammalian expression vectors (e.g., those useful for expression of a desired polynucleotide in a human subject) comprise an origin of replication, a suitable promoter and enhancer, and/or any necessary ribosome binding sites, polyadenylation sites, splice donor and acceptor sites, transcriptional termination sequences, and 5′ flanking non-transcribed sequences. In certain embodiments, the DNA sequence in the expression vector is operatively linked to an appropriate expression control sequence(s) (promoter) to direct mRNA synthesis. In some embodiments, transcription from a vector in higher eukaryotes is increased by inserting an enhancer sequence into the vector. Enhancers are cis-acting elements of DNA, usually about from 10 to 300 bp that act on a promoter to increase its transcription. In other embodiments, the expression vector also contains a ribosome binding site for translation initiation and a transcription terminator. In still other embodiments, the vector may also include appropriate sequences for amplifying expression.

### B. Therapeutic Peptides/Polypeptides

In some embodiments, rather than administering polynucleotides encoding active polypeptides to restore normal (e.g., non-PAH producing) ATP/adenosine homeostasis, polypeptides comprising the desired enzymatic activities (e.g., dephosphrylation of ATP, ADP, and/or AMP) are administered directly to a subject suffering from PAH.

As addressed above for polynucleotides, the therapeutic polypeptide may be endogenous to the subject (e.g., CD73, CD39, etc.), a variant of an endogenous protein (e.g., having sequence identity or similarity to an endogenous protein or a fragment thereof), or may be an exogenous protein sequence.

In some embodiments, a therapeutic polypeptide comprises substitutions, additions or deletions that provide for therapeutically effective molecules. In one aspect, the therapeutic polypeptides have a primary amino acid sequence in which functionally equivalent amino acid residues are substituted for residues within the sequence. Such therapeutic polypeptides retain some or all of the bioactivity of the original polypeptide sequence (e.g., SEQ ID NO: 5, SEQ ID NO: 6, or another therapeutically useful sequence), but with enhance characteristics for therapeutic administration.

For example, one or more amino acid residues of a therapeutic polypeptide may be substituted by another amino acid of a similar polarity that acts as a functional equivalent, resulting in a silent alteration. Conservative substitutions for an amino acid within therapeutic polypeptides may be selected from other members of the class to which the amino acid belongs (e.g., conservative or semi-conservative substitution).

In some embodiments, therapeutic polypeptides are modified to increase their hydrophobicity in order to enhance their penetration into the cell through the cell membrane. This can be accomplished by the addition of one or more hydrophobic amino acid residues at either the amino terminus, carboxyl terminus or within the amino acid sequence of the therapeutic polypeptide. The therapeutic polypeptide can also have one or more hydrophobic residues within its sequence replacing non-hydrophobic residues. Alternatively, in some embodiments, hydrophobic residues are replaced to increase the solubility of the polypeptide (e.g., to enhance the capacity of the enzyme to function extracellularly).

In some embodiments, one or more native amino acid residues from the therapeutic polypeptide is replaced with a non-classical amino acid residue. Examples of non-classical amino acids include: D-isomers of the common amino acids, 2,4-diaminobutyric acid, α-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, β-alanine, fluoro-amino acids, designer amino acids such as γ-methyl amino acids, Cα-methyl amino acids, Nα-methyl amino acids, and amino acid analogs in general.

Also included within the scope of the invention are therapeutic polypeptides which have been differentially modified during or after synthesis, e.g., by benzylation, glycosylation, acetylation, phosphorylation, amidation, PEGylation, derivatization by known protecting/blocking groups, proteolytic cleavage, linkage to an antibody molecule or other cellular ligand, etc. In another embodiment, the therapeutic polypeptides are conjugated to polymers, e.g., polymers known in the art to facilitate oral delivery, decrease enzymatic degradation, increase solubility of the polypeptides, or otherwise improve the chemical properties the therapeutic polypeptides for administration to humans or other animals. The polymers may be joined to the therapeutic polypeptides by hydrolyzable bonds, so that the polymers are cleaved in vivo to yield the activetherapeutic polypeptides.

In some embodiments, therapeutic polypeptides are produced in reverse order, substituting D-amino acids for the naturally occurring L-amino acids in order to increase stability and in vivo half-life on the polypeptide. In this embodiment, the amino-terminus amino acid of the therapeutic polypeptide becomes the carboxy-terminus amino acid and the carboxy-terminus amino acid becomes the amino-terminus amino acid. In some embodiments, therapeutic polypeptides are circularly permuted.

In some embodiments, therapeutic polypeptides are treated or conditioned prior to use. For example, therapeutic polypeptides may be incubated with a delivery vehicle, such as lipoproteins, nanoparticles, liposomes, etc., prior to use.

### C. Antibody Therapies

In some embodiments provided herein are isolated antibodies or antibody fragments (e.g., FAB fragments), for example, to target a particular component responsible for the dysregulation of purinergic signaling that leads to and/or is found in PAH (e.g., excess ATP, insufficient adenosine) and/or a receptor or other agent that mediates the effect of excess ATP and/or insufficient adenosine (e.g., P2X1).

In some embodiments, antibodies find use as agents to alter signal transduction. In some embodiments, specific antibodies that bind to the binding domains of ATP, ADP, AMP, and/or adenosine receptors or other proteins involved in signaling are used to inhibit the interaction between the various proteins and their interaction with their ligands. In some embodiments, antibodies are used therapeutically to inhibit interactions in signal transduction pathways leading to the various physiological and cellular PAH.

Antibodies may be prepared using various immunogens. In one embodiment, the immunogen is a P2X1 peptide to generate antibodies that recognize human P2X1. Other immunogens may be used to generate antibodies to other components of the dysregulated pathways described herein (e.g., P2X2, P2X3, P2X4, P2X5, P2Y1, P2Y2, P2Y4, P2Y6, P2Y12, ADORA1, ADORA2A, ADORA2B, and ADORA3). Such antibodies include, but are not limited to polyclonal, monoclonal, chimeric, single chain, Fab fragments, Fab expression libraries, or recombinant (e.g., chimeric, humanized, etc.) antibodies. Antibodies can be produced by using a protein of the present invention as the antigen according to a conventional antibody or antiserum preparation process.

Various procedures known in the art may be used for the production of polyclonal antibodies. For the production of antibody, various host animals can be immunized by injection with the epitope including but not limited to rabbits, mice, rats, sheep, goats, etc. In a preferred embodiment, the peptide is conjugated to an immunogenic carrier (e.g., diphtheria toxoid, bovine serum albumin (BSA), or keyhole limpet hemocyanin (KLH)). Various adjuvants may be used to increase the immunological response, depending on the host species, including but not limited to Freund's (complete and incomplete), mineral gels (e.g., aluminum hydroxide), surface active substances (e.g., lysolecithin, pluronic polyols, polyanions, peptides, oil emulsions, keyhole limpet hemocyanins, dinitrophenol, and potentially useful human adjuvants such as BCG (Bacille Calmette-Guerin) and Corynebacterium parvum).

For preparation of monoclonal antibodies, it is contemplated that any technique that provides for the production of antibody molecules by continuous cell lines in culture will find use herein (See e.g., Harlow and Lane, Antibodies: A Laboratory Manual, Cold Spring Harbor Laboratory Press, Cold Spring Harbor, N.Y.). These include but are not limited to the hybridoma technique originally developed by Kohler and Milstein (Kohler and Milstein, Nature 256:495-497 [1975]), as well as the trioma technique, the human B-cell hybridoma technique (See e.g., Kozbor et al., Immunol. Tod., 4:72 [1983]), and the EBV-hybridoma technique to produce human monoclonal antibodies (Cole et al., in Monoclonal Antibodies and Cancer Therapy, Alan R. Liss, Inc., pp. 77-96 [1985]). In an additional embodiment of the invention, monoclonal antibodies are produced in germ-free animals utilizing technology such as that described in PCT/US90/02545). Furthermore, it is contemplated that human antibodies will be generated by human hybridomas (Cote et al., Proc. Natl. Acad. Sci. USA 80:2026-2030 [1983]) or by transforming human B cells with EBV virus in vitro (Cole et al., in Monoclonal Antibodies and Cancer Therapy, Alan R. Liss, pp. 77-96 [1985]).

In addition, it is contemplated that techniques described for the production of single chain antibodies (U.S. Pat. No. 4,946,778; herein incorporated by reference) will find use in producing target specific single chain antibodies. An additional embodiment of the invention utilizes the techniques described for the construction of Fab expression libraries (Huse et al., Science 246:1275-1281 [1989]) to allow rapid and easy identification of monoclonal Fab fragments with the desired specificity.

It is contemplated that any technique suitable for producing antibody fragments will find use in generating antibody fragments that contain the idiotype (antigen binding region) of the antibody molecule. For example, such fragments include but are not limited to: F(ab′)2 fragment that can be produced by pepsin digestion of the antibody molecule; Fab′ fragments that can be generated by reducing the disulfide bridges of the F(ab′)2 fragment, and Fab fragments that can be generated by treating the antibody molecule with papain and a reducing agent.

### D. Gene Therapy

In some embodiments, provided herein are methods and compositions suitable for gene therapy to alter expression, production, and/or function of targets responsible for, involved in, and/or effected by the purinergic dysregulation described herein (e.g., CD39, CD73, P2X1, P2X2, P2X3, P2X4, P2X5, P2Y1, P2Y2, P2Y4, P2Y6, P2Y12, ADORA1, ADORA2A, ADORA2B, and ADORA3). In some embodiments, gene therapy is performed by providing a subject with an allele of a gene that is free of PAH causing polymorphisms or mutations.

Viral vectors commonly used for in vivo or ex vivo targeting and therapy procedures are DNA-based vectors and retroviral vectors. Methods for constructing and using viral vectors are known in the art (See e.g., Miller and Rosman, BioTech., 7:980-990 [1992]). Preferably, the viral vectors are replication defective, that is, they are unable to replicate autonomously in the target cell. In general, the genome of the replication defective viral vectors that are used within the scope of the present invention lack at least one region that is necessary for the replication of the virus in the infected cell. These regions can either be eliminated (in whole or in part), or be rendered non-functional by any technique known to a person skilled in the art. These techniques include the total removal, substitution (by other sequences, in particular by the inserted nucleic acid), partial deletion or addition of one or more bases to an essential (for replication) region. Such techniques may be performed in vitro (i.e., on the isolated DNA) or in situ, using the techniques of genetic manipulation or by treatment with mutagenic agents.

In some embodiments, the replication defective virus retains the sequences of its genome that are necessary for encapsidating the viral particles. DNA viral vectors include an attenuated or defective DNA viruses, including, but not limited to, herpes simplex virus (HSV), papillomavirus, Epstein Barr virus (EBV), adenovirus, adeno-associated virus (AAV), and the like. Use of defective viral vectors allows for administration to cells in a specific, localized area, without concern that the vector can infect other cells. Thus, a specific tissue can be specifically targeted.

In some embodiments, for in vivo administration, an appropriate immunosuppressive treatment is employed in conjunction with the viral vector (e.g., adenovirus vector), to avoid immuno-deactivation of the viral vector and transfected cells. For example, immunosuppressive cytokines, such as interleukin-12 (IL-12), interferon-gamma (IFN-.gamma.), or anti-CD4 antibody, can be administered to block humoral or cellular immune responses to the viral vectors. In addition, it is advantageous to employ a viral vector that is engineered to express a minimal number of antigens.

Another technique for gene therapy include vector introduction by lipofection (Felgner et. al., Proc. Natl. Acad. Sci. USA 84:7413-7417 [1987]; See also, Mackey, et al., Proc. Natl. Acad. Sci. USA 85:8027-8031 [1988]; Ulmer et al., Science 259:1745-1748 [1993]). The use of cationic lipids may promote encapsulation of negatively charged nucleic acids, and also promote fusion with negatively charged cell membranes (Felgner and Ringold, Science 337:387-388 [1989]). Particularly useful lipid compounds and compositions for transfer of nucleic acids are described in WO95/18863 and WO96/17823, and in U.S. Pat. No. 5,459,127, herein incorporated by reference.

Other molecules are also useful for facilitating transfection of a nucleic acid in vivo, such as a cationic oligopeptide (e.g., WO95/21931), peptides derived from DNA binding proteins (e.g., WO96/25508), or a cationic polymer (e.g., WO95/21931).

It is also possible to introduce the vector in vivo as a naked DNA plasmid. For example, methods for formulating and administering naked DNA to mammalian muscle tissue are disclosed in U.S. Pat. Nos. 5,580,859 and 5,589,466, both of which are herein incorporated by reference.

DNA vectors for gene therapy can be introduced into a subject (e.g., a subject's cells) by methods known in the art, including but not limited to transfection, electroporation, microinjection, transduction, cell fusion, DEAE dextran, calcium phosphate precipitation, use of a gene gun, or use of a DNA vector transporter (See e.g., Wu et al., J. Biol. Chem., 267:963 [1992]; Wu and Wu, J. Biol. Chem., 263:14621 [1988]; and Williams et al., Proc. Natl. Acad. Sci. USA 88:2726 [1991]). Receptor-mediated DNA delivery approaches can also be used (Curiel et al., Hum. Gene Ther., 3:147 [1992]; and Wu and Wu, J. Biol. Chem., 262:4429 [1987]).

### E. Pharmaceutical Therapy

Some embodiments herein pertain to the use pharmaceutical agents (e.g., small molecules, peptides, antibodies, etc.) in the treatment of PAH. Experiments conducted during development of embodiments herein have demonstrated multiple targets for the treatment of PAH which are targetable by administration of a therapeutic agent. For example, an inhibitor of P2X1 activity (e.g., P2X1 antagonist), an inhibitor of ATP biosynthesis (e.g., ATP synthase antagonist), a stable analogue of adenosine, etc. each address different aspects of purinergic dysregulation that the experiments conducted during development of embodiments herein have demonstrated to by linked to and/or causative of PAH and/or PAH symptoms.

Multiple P2X1 inhibitors are known, including:

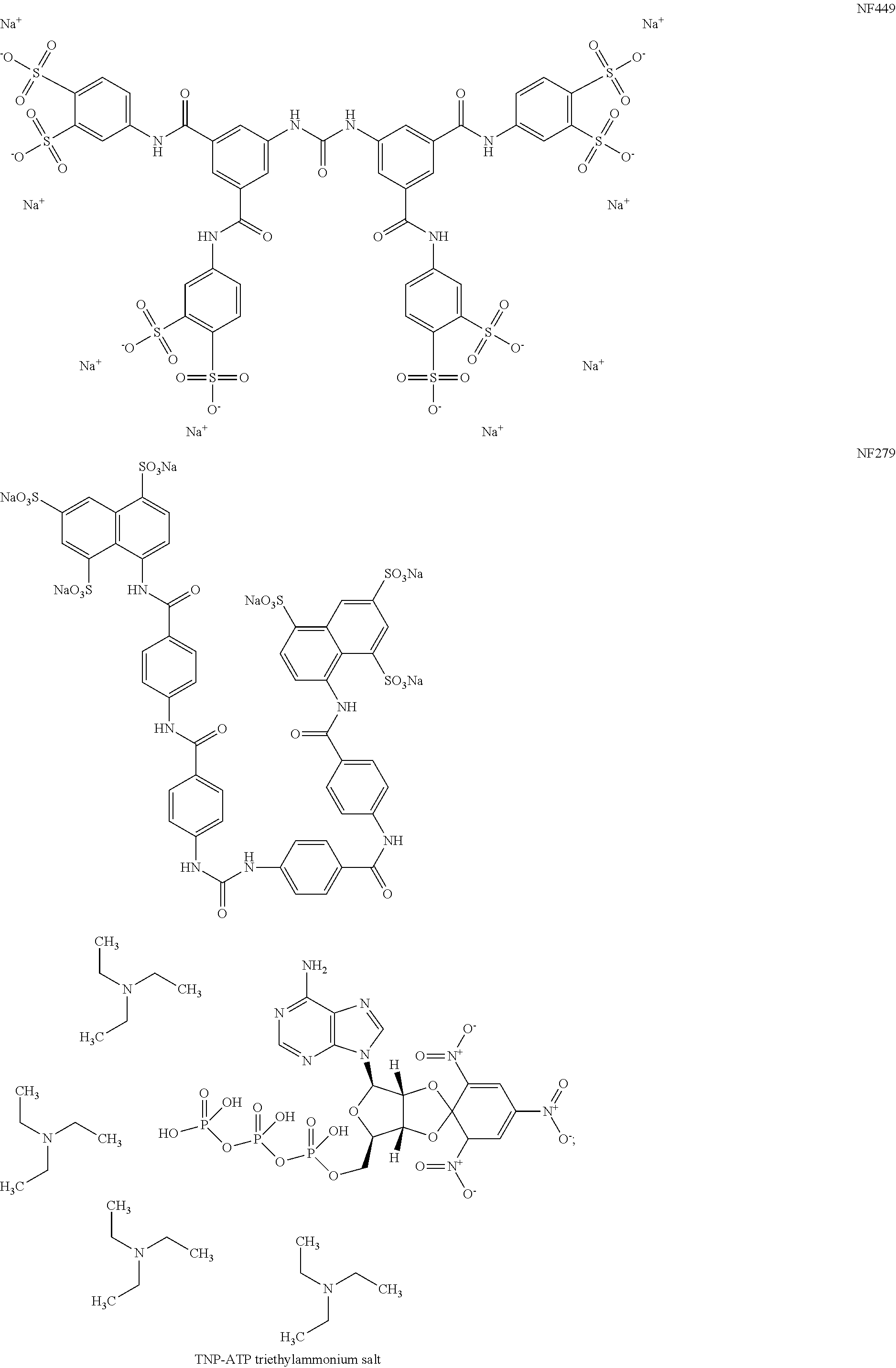

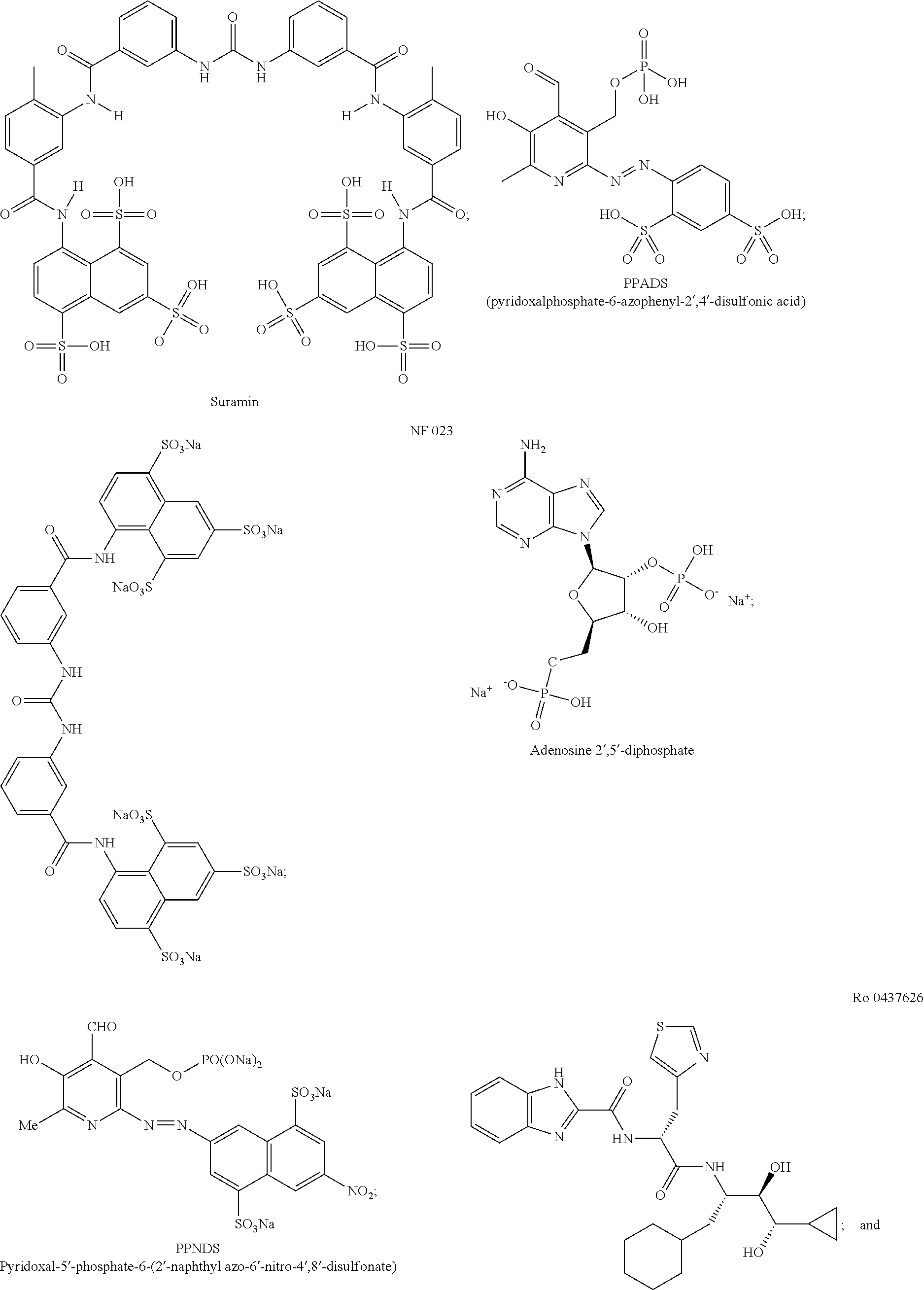

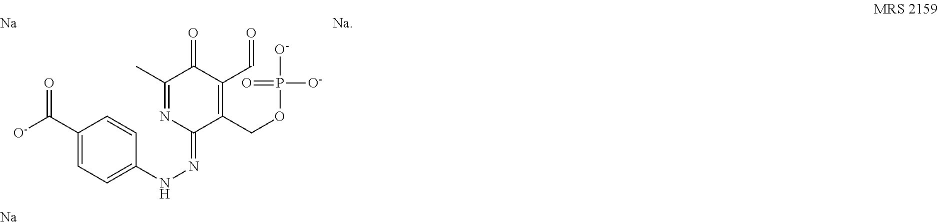

Administration of these or other P2X1 inhibitors finds use in the reduction of P2X1 signaling that results from increased extracellular ATP concentrations and overexpression of P2X1, and causes PAH and/or PAH-associated symptoms.

Over 250 natural and synthetic inhibitors of ATP synthase have been identified (Hong and Pedersen, Microbiol Mol Biol Rev. 2008 December; 72(4): 590-641.; incorporated by reference in its entirety). In some embodiments, targeting of ATP synthase with therapeutic inhibitory agents reduces the production of ATP and reduces extracellular ATP concentration.

In some embodiments, treatment of PAH with adenosine has historically been ineffective, likely due to the instability of extracellular adenosine. Therefore, in some embodiments, the recued extracellular levels of adenosine that are identified in experiments conducted during development of embodiments herein to be linked to PAH and PAH-associated symtoms, are treated by the administration of stable analogues of adenosine. Various adenosine analogues include:

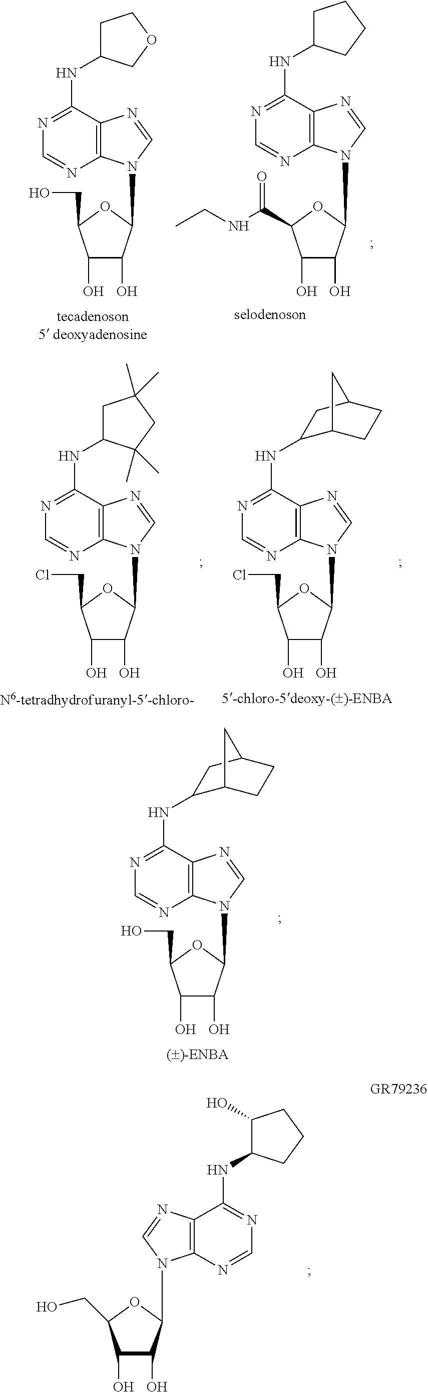

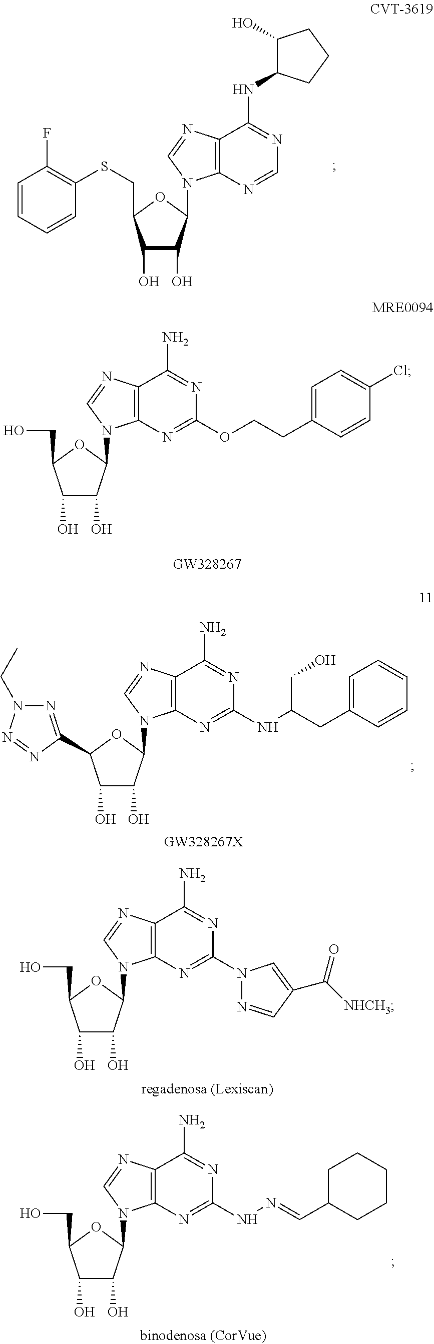

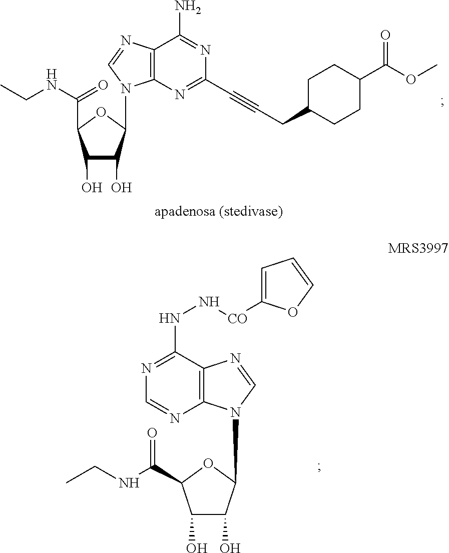

and other adenosine analogues, for example, described in Samsel and Dzierzbick, Pharmacol Rep. 2011; 63(3):601-17.; incorporated by reference in its entirety).

In some embodiments, provided herein are pharmaceutical compositions comprising a therapeutic agent (e.g., P2X1 inhibitor, adenosine analogue, etc.), alone or in combination with at least one other non-therapeutic agent, such as a stabilizing compound, and may be administered in any sterile, biocompatible pharmaceutical carrier, including, but not limited to, saline, buffered saline, dextrose, and water.

As is well known in the medical arts, dosages for any one patient depends upon many factors, including the patient's size, body surface area, age, the particular compound to be administered, sex, time and route of administration, general health, and interaction with other drugs being concurrently administered.

Pharmaceutical compositions may be formulated and administered systemically or locally. Techniques for formulation and administration may be found in the latest edition of “Remington's Pharmaceutical Sciences” (Mack Publishing Co, Easton Pa.). Suitable routes may, for example, include oral or transmucosal administration; as well as parenteral delivery, including intramuscular, subcutaneous, intramedullary, intrathecal, intraventricular, intravenous, intraperitoneal, or intranasal administration.

For injection, pharmaceutical compositions may be formulated in aqueous solutions, preferably in physiologically compatible buffers such as Hanks' solution, Ringer's solution, or physiologically buffered saline. For tissue or cellular administration, penetrants appropriate to the particular barrier to be permeated are used in the formulation. Such penetrants are generally known in the art.

In other embodiments, the pharmaceutical compositions are formulated using pharmaceutically acceptable carriers well known in the art in dosages suitable for oral administration. Such carriers enable the pharmaceutical compositions to be formulated as tablets, pills, capsules, liquids, gels, syrups, slurries, suspensions and the like, for oral or nasal ingestion by a patient to be treated.

Pharmaceutical compositions include compositions wherein the active ingredients (e.g., P2X1 inhibitor, adenosine analog, etc.) are contained in an effective amount to achieve the intended purpose. For example, an effective amount of therapeutic may be that amount that restores a normal (non-diseased) ATP levels, adenosine levels, and/or P2X1 signaling. Determination of effective amounts is well within the capability of those skilled in the art, especially in light of the disclosure provided herein.

In addition to the active therapeutic ingredients, pharmaceutical compositions may contain suitable pharmaceutically acceptable carriers comprising excipients and auxiliaries that facilitate processing of the active compounds into preparations that can be used pharmaceutically. The preparations formulated for oral administration may be in the form of tablets, dragees, capsules, or solutions. The pharmaceutical compositions of the present invention may be manufactured in a manner that is itself known (e.g., by means of conventional mixing, dissolving, granulating, dragee-making, levigating, emulsifying, encapsulating, entrapping or lyophilizing processes).

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions.

Pharmaceutical preparations for oral use can be obtained by combining the active compounds with solid excipient, optionally grinding a resulting mixture, and processing the mixture of granules, after adding suitable auxiliaries, if desired, to obtain tablets or dragee cores. Suitable excipients are carbohydrate or protein fillers such as sugars, including lactose, sucrose, mannitol, or sorbitol; starch from corn, wheat, rice, potato, etc; cellulose such as methyl cellulose, hydroxypropylmethyl-cellulose, or sodium carboxymethylcellulose; and gums including arabic and tragacanth; and proteins such as gelatin and collagen. If desired, disintegrating or solubilizing agents may be added, such as the cross-linked polyvinyl pyrrolidone, agar, alginic acid or a salt thereof such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound, (e.g., dosage).

Pharmaceutical preparations for oral administration include push-fit capsules made of gelatin, as well as soft, sealed capsules made of gelatin and a coating such as glycerol or sorbitol. The push-fit capsules can contain the active ingredients mixed with a filler or binders such as lactose or starches, lubricants such as talc or magnesium stearate, and, optionally, stabilizers. In soft capsules, the active compounds may be dissolved or suspended in suitable liquids, such as fatty oils, liquid paraffin, or liquid polyethylene glycol with or without stabilizers.

Therapeutic compositions formulated in a pharmaceutical acceptable carrier may be prepared, placed in an appropriate container, and labeled for treatment of the indicated condition (e.g., PAH).

The pharmaceutical composition may be provided as a salt and can be formed with many acids, including but not limited to hydrochloric, sulfuric, acetic, lactic, tartaric, malic, succinic, etc. Salts tend to be more soluble in aqueous or other protonic solvents that are the corresponding free base forms. In other cases, the preferred preparation may be a lyophilized powder in 1 mM-50 mM histidine, 0.1%-2% sucrose, 2%-7% mannitol at a pH range of 4.5 to 5.5 that is combined with buffer prior to use.

In some embodiments, a therapeutically effective dose may be estimated initially from cell culture assays and/or animal models (particularly murine models). A therapeutically effective dose refers to that amount that effectively addresses and underlying cause and/or ameliorates symptoms of the disease state or unwanted condition (e.g., PAH). Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index, and it can be expressed as the ratio LD₅₀/ED₅₀. Compounds that exhibit large therapeutic indices are preferred. Data obtained from these cell culture assays and additional animal studies can be used in formulating a range of dosage for human use. The dosage of such compounds lies preferably within a range of circulating concentrations that include the ED₅₀ with little or no toxicity. The dosage varies within this range depending upon the dosage form employed, sensitivity of the patient, and the route of administration. The exact dosage is chosen by the individual clinician in view of the patient to be treated. Dosage and administration are adjusted to provide sufficient levels of the active moiety or to maintain the desired effect. Additional factors which may be taken into account include the severity of the disease state; age, weight, and gender of the patient; diet, time and frequency of administration, drug combination (s), reaction sensitivities, and tolerance/response to therapy. Long acting pharmaceutical compositions might be administered every 3 to 4 days, every week, or once every two weeks depending on half-life and clearance rate of the particular formulation.

Typical dosage amounts may vary from 0.1 to 100,000 micrograms, up to a total dose of about 1 g, depending upon the route of administration. Guidance as to particular dosages and methods of delivery is provided in the literature (See, U.S. Pat. Nos. 4,657,760; 5,206,344; 5,225,212; WO2004/097009, or WO2005/075465, each of which are herein incorporated by reference).

### F. RNA Interference

As described herein, in some embodiments, overexpression or over activity (e.g., due to excess substrate (e.g., ATP) of one or more proteins (e.g., P2X1) is implicated (e.g., causative) in PAH and/or PAH symptoms. Therefore, in some embodiments, it is desirable to reduce expression of such proteins (e.g., P2X1).

RNAi represents an evolutionary conserved cellular defense for controlling the expression of foreign genes in most eukaryotes, including humans. RNAi is triggered by double-stranded RNA (dsRNA) and causes sequence-specific mRNA degradation of single-stranded target RNAs homologous in response to dsRNA. The mediators of mRNA degradation are small interfering RNA duplexes (siRNAs), which are normally produced from long dsRNA by enzymatic cleavage in the cell. siRNAs are generally approximately twenty-one nucleotides in length (e.g. 21-23 nucleotides in length), and have a base-paired structure characterized by two nucleotide 3′-overhangs. Following the introduction of a small RNA, or RNAi, into the cell, the sequence is delivered to the RNA-induced silencing complex (RISC). RISC recognizes the target and cleaves it with an endonuclease. It is noted that if larger RNA sequences are delivered to a cell, RNase III enzyme (Dicer) converts longer dsRNA into 21-23 nt ds siRNA fragments.

Chemically synthesized siRNAs are powerful gene-specific therapeutic agents (Tuschl and Borkhardt, Molecular Intervent. 2002; 2(3):158-67, herein incorporated by reference). The transfection of siRNAs into subject cells results in the potent, long-lasting post-transcriptional silencing of specific genes (Caplen et al, Proc Natl Acad Sci U.S.A. 2001; 98: 9742-7; Elbashir et al., Nature. 2001; 411:494-8; Elbashir et al., Genes Dev. 2001; 15: 188-200; and Elbashir et al., EMBO J. 2001; 20: 6877-88, all of which are herein incorporated by reference). Methods and compositions for performing RNAi with siRNAs are described, for example, in U.S. Pat. No. 6,506,559, herein incorporated by reference. siRNAs are extraordinarily effective at lowering the amounts of targeted RNA, and by extension proteins, frequently to undetectable levels. The silencing effect can last several months, and is extraordinarily specific, because one nucleotide mismatch between the target RNA and the central region of the siRNA is frequently sufficient to prevent silencing Brummelkamp et al, Science 2002; 296:550-3; and Holen et al, Nucleic Acids Res. 2002; 30:1757-66, both of which are herein incorporated by reference.

In some embodiments, RNAi for inhibiting the expression of one or more endogenous proteins (e.g., P2X1 or other ATP-responsive proteins (e.g., receptors)) is provided. Design of siRNA targeting endogenous proteins (e.g., P2X1 or other ATP-responsive proteins (e.g., receptors)) is within the knowledge in the field. For example, Oligoengine's web page provides a design tool that finds RNAi candidates based on Elbashir's (Elbashir et al, Methods 2002; 26: 199-213, herein incorporated by reference) criteria. Other design tools may also be used, such as the Cenix Bioscience design tool offered by Ambion. In addition, there is also the Si2 silencing duplex offered by Oligoengine.

In some embodiments, siRNA are administered to a subject using compositions and methods described herein for the administration or polynucleotides and/or gene therapy reagents.

### G. Combination Therapies

In some embodiments, the therapies disclosed herein are combined or used in combination with other agents useful in the treatment of PAH. Or, by way of example only, the therapeutic effectiveness of one of the therapies described herein may be enhanced by administration of an adjuvant (e.g., by itself the adjuvant may only have minimal therapeutic benefit, but in combination with another therapeutic agent, the overall therapeutic benefit to the patient is enhanced).

Such other agents, adjuvants, or drugs, may be administered, by a route and in an amount commonly used therefor, simultaneously or sequentially with a compound as disclosed herein. When a compound as disclosed herein is used contemporaneously with one or more other drugs, a pharmaceutical composition containing such other drugs in addition to the compound disclosed herein may be utilized, but is not required.

In some embodiments, one or more of the therapies provided herein are combined with each other, and/or with known treatments for PAH.

Existing treatments for PAH include: optimization of left ventricular function by the use of, for example, diuretics, digoxins, blood thinners, and/or repair/replacement the mitral valve or aortic valve; high dose calcium channel blockers, vasoactive substances, such as, endothelin receptor antagonists, phosphodiesterase type 5 (PDE-5) inhibitors, prostacyclin derivatives, etc.; prostaglandins/prostanoids, such as prostacyclin (prostaglandin I₂), Epoprostenol (synthetic prostacyclin), Treprostinil, Remodulin, Iloprost, etc.; endothelin receptor antagonists, such as, bosentan, Sitaxentan (Thelin), ambrisentan, etc.; Phosphodiesterase type 5 inhibitors, such as, sildenafil, Tadalafil, etc.; nitric oxide pathway antagonists, such as, soluble guanylate cyclase stimulators, class V phosphodiesterase inhibitors, etc.; activators of soluble guanylate cyclase, such as, cinaciguat and riociguat; etc. While these treatments have only been minimally or modestly effective, in some embodiments, synergies exist between these existing treatments and those described herein.

In some embodiments, experiments conducted during development of embodiments herein indicate that one or more of the following therapeutics may find use in treatment of PAH and/or PAH symptoms, such as: an anticoagulant (e.g., heparin, Coumadin, protamine, hirudin, etc.), an antithrombotic agent (e.g., clopidogrel, heparin, hirudin, iloprost, etc.), an antiplatelet agent (e.g., aspirin, dipyridamole, etc.), an anti-inflammatory agent (e.g., methylprednisolone, dexamethasone, tranilast, etc.), an anti-proliferative/immunosuppressive agent (e.g., trapidil, tyrphostin, rapamycin, FK-506, mycophenolic acid), a cytostatic drug (e.g., paclitaxel, rapamycin, rapamycin analogs (e.g., everolimus, tacrolimus, etc.), etc.), an antioxidant (e.g., probucol, vitamin C, retinoids, resveratrol, etc.), etc. In some embodiments, these therapeutics are co-administered with one of the other treatments (e.g., nucleotidease, purinergic receptor antagonist, adenosine receptor agonist, etc.) indicated and described herein.

In some embodiments, one or more therapeutic approaches described herein and/or existing therpeis are co-administered to a subject. In some embodiments, co-administration involves co-formulation of two or more agents together into the same medicament. In other embodiments, the agents are in separate formulations but are administered together, either simultaneously or in sequence (e.g., separated by one or more minutes, hours, days, etc.). In some embodiments, where a synergistic or additive benefit is achieved, the co-administered agent may be provided at a lower dose than would normally be administered if that agent were being used in isolation to treat the disease or condition.

### H. Kits

The technology provided herein also includes kits for use in the instant methods. Kits of the technology comprise one or more containers comprising a therapeutic approach dewcribed herein and/or a second agent, and in some varations further comprise instructions for use in accordance with any of the methods provided herein. The kit may further comprise a description of selecting an individual suitable treatment. Instructions supplied in the kits of the technology are typically written instructions on a label or package insert (e.g., a paper insert included with the kit), but machine-readable instructions (e.g., instructions carried on a magnetic or optical storage disk) are also contemplated. In some embodiments, the kit is a package containing a sealed container comprising any one of the preparations described above, together with instructions for use. The kit can also include a diluent container containing a pharmaceutically acceptable diluent. The kit can further comprise instructions for mixing the preparation and the diluent. The diluent can be any pharmaceutically acceptable diluent. Well known diluents include 5% dextrose solution and physiological saline solution. The container can be an infusion bag, a sealed bottle, a vial, a vial with a septum, an ampoule, an ampoule with a septum, an infusion bag, or a syringe. The containers can optionally include indicia indicating that the containers have been autoclaved or otherwise subjected to sterilization techniques. The kit can include instructions for administering the various solutions contained in the containers to subjects.

### EXPERIMENTAL

### Example 1

### Materials and Methods

### Procurement of Human Lung Tissue

Formalin-fixed, paraffin embedded human lung tissue from controls and patients with IPAH were obtained through the Cardiovascular Medical Research and Education Fund—Pulmonary Hypertension Breakthrough Initiative (PHBI) network. The PHBI study protocol was approved by the Institutional Review Boards of the participating sites in the network, and all sites were adherent to the requirements of the U.S. Federal Policy for the Protection of Human Subjects (45 CFR, Part 46), and supported the general ethical principles of the Declaration of Helsinki.

### Immunohistochemistry and Immunofluorescence Microscopy

Formalin-fixed, paraffin-embedded lungs were sectioned at a thickness of 5 μm. Sections underwent deparaffinization, dehydration, antigen retrieval and quenching of endogenous peroxidase activity. Blocking, primary antibody labeling and immunoperoxidase staining were performed as recommended by the ImmPRESS Polymer Detection Kit and ImmPACT DAB peroxidase HRP substrate (Vector Laboratories, Burlingame, Calif.). Rabbit anti-human CD39 antibody (sc-33558, Santa Cruz Biotechnology, Dallas, Tex.) and guinea pig anti-mouse NTPDase1 antibody (ectonucleotidases-ab.com, Quebec, Canada) were used to detect CD39 in human and mouse lung tissue, respectively. Rabbit P2X1 receptor antibody (H-100, Santa Cruz Biotechnology, Dallas, Tx) was for both mouse and human lung sections. Quantification of vessel CD39 staining was calculated as the percent of CD31-DAB-positive endothelial area that also stained positive for CD39. The area of positive antibody staining was determined using the color devonvolution followed by threshold functions within Fiji (48) (Image J, NIH, Bethesda, Md.). Percent CD39 staining for 5 vessels from each group was calculated as: (area of CD39-positive staining/area of CD31-positive staining)*100.

Assessment of mouse pulmonary arterial medial thickness was performed following αSMA primary antibody (ab5694, Abcam, Cambridge, Mass.) staining and immunofluorescence labeling application with the Tyramide Signal Amplification Plus System (PerkinElmer, Waltham, Mass.). Sections were then visualized using at 40× or 60× magnification using a Nikon TE2000E2 microscope (Melville, N.Y.) with MetaMorph software (Molecular Devices, Sunnyvale, Calif.). Images were captured and ImageJ (NIH, Bethesda, Md.) was used to measure overall vessel and medial diameter. Percent of media thickness for each vessel was computed as: (external diameter-internal diameter/external diameter)*100. Presented values are the mean of 10 fields from 6 mice in each group. Vessels were categorized based on diameter (<50 μm, 51-100 μm, >100 μm). All vessels were analyzed by an investigator blinded to study conditions.

### Generation of CD39-Deficient Mice

All animal experiments were approved by, and carried out in accordance with the University of Michigan University Committee on Use and Care of Animals (UCUCA) guidelines. CD39-deficient mice (CD39^{-/-}) were generated from C57BL/6 mice through the University of Michigan Transgenic Animal Model Core by deleting the first exon of CD39 using Cre-Lox recombination. A ploxPFlpneo cloning vector containing two loxP sites flanking the first exon of CD39. BACPAC clone RP23-117D11 (derived from a C57BL/6 mouse) was used as source DNA for the insertion of BamHI sites at −490 and +950 of exon 1. This vector was introduced into C57BL/6-derived Bruce 4 embryonic stem (ES) cells and selected in G418. ES clones with successful insertion of LoxP were identified by qPCR, confirmed by Southern blot analyses of Bgl1/Sal1-digested DNA, and injected into C57BL/6 blastocysts. Probes were designed to detect the 5′ end and 3′ ends of the DNA targeted for homologous recombination in transfected stem cell lines. The 5′ and 3′ probes were radiolabeled and hybridized with genomic DNA purified from untransfected stem cells to confirm a successfully transfected stem cell line, and an F1 generation mouse. High contribution from the ES cell clone was assessed using coat color contribution. Successive breeding to a FLP recombinase-expressing mouse followed by a ubiquitously-expressed Ella-CRE-recombinase-expressing mouse was used to ablate CD39. Homozygous animals were produced from the mating of hemizygous chimera offspring. Genotyping by PCR analysis of genomic DNA from tail tips was performed on all mice using primer sets to confirm wild-type (WT) CD39 (5′-TGGGAAGGG GTCAGCTCTATGTGGTA-3′ (SEQ ID NO: 1) and 5′-CCTTCCCCTTCCTTCCTC TTTTCCTCCGTTAT-3′ (SEQ ID NO: 2)) or knockout (KO) CD39 genotype (5′-GTCATTTCACAGCTGGCA AGAGGTA-3′ (SEQ ID NO: 3) and 5′-CAGGAAGTGGAGGTGATAGGGACAACA-3′ (SEQ ID NO: 4)). Quantitative RT-PCR analyses were used to assess CD39 mRNA in various adult mouse organs. Wild-type mice (CD39^{+/+}) of the same genetic background (C57BL/6) were purchased from Jackson Laboratory (Bar Harbor, Me.). All mice were housed in a designated Animal Resource Facility at the University of Michigan under specific pathogen-free conditions.

### Western Blot Analyses

Lung tissues from normoxic wild-type (CD39^{+/+}) and knockout (CD39^{-/-}) mice were homogenized (gentleMACS Dissociator and tubes, Miltenyi Biotec, Bergisch Gladbach, Germany). Protein was isolated using lysis buffer containing 1 mM Tris-HCl, pH 7.4, 0.5M EDTA, 5M NaCl, 1% TritonX-100, 1% sodium deoxycholate and 10% SDS supplemented with protease inhibitors (Complete 185 Protease Inhibitor Cocktail, Roche, Indianapolis, Ind.). Western blotting was carried out using standard protocols using sheep anti-mouse CD39 (AF4398, R&D Systems, Minneapolis, Minn.) and rabbit anti-mouse GAPDH (ABS16, Millipore, Billerica, Mass.) antibodies followed by ECL chemiluminescence substrate (Thermo Scientific, Rockford, Ill.). Final blots were imaged using a flatbed scanner.

### Normobaric Hypoxia and Normoxia Exposure

Seven-week-old CD39^{+/+} and CD39^{-/-} mice were maintained at 10% oxygen (normobaric) for four weeks using a purpose-built, normobaric hypoxia chamber with automated oxygen-regulating capability. Circulating air was scavenged using charcoal and soda lime (Sigma-Aldrich, St. Louis, Mo.). Temperature and humidity were monitored, and the latter maintained within normal limits using Dririte (Sigma-Aldrich, St. Louis, Mo.). Normoxic control mice were maintained in the same room at 21% oxygen, which was verified by a continuous oxygen sensor (BioSpherix, Lacona, N.Y.). All animals were given free access to food and water, and were maintained on a 12-hour dark/light cycle.

### Hemodynamic Measurements

Mice were anesthetized using 2% isoflurane. A tracheostomy was performed, and animals were ventilated using 21% or 10% oxygen administered thorough the ventilator circuit. Optimal ventilator settings were confirmed using arterial blood gas measurements. After a median sternotomy was performed, hemodynamic measurements were made using a 1.2F solid state pressure catheter (Scisense Transonic, London, ON) inserted through the right ventricular (RV) free wall into the RV cavity and advanced into the pulmonary artery (PA). Placement was verified using the pressure waveform. Data including the right ventricular systolic pressure (RVSP) and mean PA (mPA) pressures were collected and analyzed using LabScribe2 (iWorx, Dover, N.H.). Continuous EKG monitoring was performed using the MouseMonitor (Indus Instruments, Webster, Tex.).

### Blood Collection

Plasma for ATP, ADP, AMP and adenosine analysis was obtained by puncturing the right ventricle with a 26-gauge needle and drawing 500 μL of whole blood into a syringe pre-filled with 500 μL of chilled “stop solution.” The “stop solution” used was based upon prior studies (18, 42) and contained 4.15 mM EDTA (arrests ATP catabolism), 5 nM NBTI (inhibits ATP release from erythrocytes), 10 μM forskolin (stabilizes platelets to prevent ATP release), 100 μM IBMX (inhibits cAMP phosphodiesterase), 40 μM dipyridamole (inhibits adenosine reuptake and adenosine deaminase), 10 μM EHNA (inhibits adenosine deaminase) and 10 μM 5-iodotubericidin (inhibits adenosine kinase). All reagents were obtained from Sigma-Aldrich (St. Louis, Mo.). The blood/stop solution mixture was centrifuged (13,000×g) for 2 minutes at 4 degrees Celsius, and the supernatant was re-centrifuged for 2 minutes. Aliquots of the final supernatant were stored at −80 degrees Celsius. Blood for arterial blood gas analysis was drawn from the left ventricle using heparinized syringes and analyzed immediately.

Arterial Blood Gas Analysis Values including the partial pressure of oxygen (pO₂) and hemoglobin concentration were measured in heparinized arterial blood drawn from the left ventricle using an ABL800 flex analyzer (Radiometer America, Carlsbad Calif.).

### Tissue Collection and Preparation

Lungs were gently perfused through the right ventricle using 20 mM EDTA in PBS at a constant pressure of 20 cm H₂O until the tissue blanched (3-5 minutes). Lungs used for histology were then perfused with 10% buffered formalin administered via the tracheostomy site, and transferred to 70% ethyl alcohol 24 hours later. Lung samples for transcriptomics and Western blot analysis were placed in Allprotect Tissue Reagent (Qiagen, Vallencia, Calif.) and stored at −80 degrees Celsius. The heart was excised for weight measurements.

### Quantitative RT-PCR Analysis of Purinergic Receptors

Taqman probes (Applied Biosystems, Grand Island, N.Y.) were used to quantitate expression of the following purinergic receptor genes in whole lung homogenates: P2X1, P2X2, P2X3, P2X4, P2X5, P2Y1, P2Y2, P2Y4, P2Y6, P2Y12, ADORA1, ADORA2A, ADORA2B, and ADORA3.

### Right Ventricular Weight and Fulton's Index

The heart was excised, the right ventricular free wall was dissected and weighed, and the remaining septum and left ventricle were weighed. Right ventricular hypertrophy was assessed using Fulton's Index: (right ventricular weight/septum+left ventricular weight)*100.

### Metabolomics Analysis

Plasma ATP, ADP, AMP and adenosine extraction was performed using a mixture of methanol, acetone and acetonitrile (1:1:1). The extraction solvent was added to plasma samples in Eppendorf tubes using a 4:1 solvent-to-sample ratio. The mixture was vortexed briefly, placed on ice for 5 minutes, vortexed again, and then centrifuged at 15,000×g for 5 minutes. Supernatant containing the metaboiltes was removed, dried and reconstituted in 100 μL of a 9:1 mixture of methanol and water. A series of calibration standards were prepared along with samples to facilitate quantification of metabolites.

An Agilent 1200 chromatography platform (Agilent Technologies, Santa Clara, Calif.) with a Luna NH2 HILIC (hydrophilic interaction chromatography) column (Phenomenex, Torrance, Calif.) was used for chromatographic separation. An Agilent 6410 series triple quadrupole mass spectrometer (Agilent Technologies, Santa Clara, Calif.) with electrospray ionization source (ESI) was operated in negative mode. The following transitions were used to identify and quantify metabolites: Adenosine: mass/charge (m/z) 266.1→m/z 134.1; AMP: m/z 346.1→m/z 79; ADP: m/z426.0→m/z 79; ATP: m/z 506.0→m/z 79. Data were processed by MassHunter workstation software, version B.04 (Agilent Technologies, Santa Clara, Calif.). Results were normalized to plasma volume and doubled (to adjust for the 1:1 dilution of whole blood into “stop solution”) to calculate final μM concentrations.

### Ectonucleotidase Reconstitution and P2×1 Receptor Blocking

Alzet osmotic pumps (DURECT, Cupertino, Calif.) were used to deliver either 15 units of

apyrase (A6410, Sigma-Aldrich, St. Louis, Mo.), 25 mM of NF279 (Tocris Bioscience/Bio-Techne, Minneapolis, Minn.) or sterile 0.9% sodium chloride over a four-week period at a rate of 0.11 μl/hr.

### Statistical Analysis

Statistical analyses and graph creation were performed using SPSS for Mac (IBM, Amnok, N.Y.). Data are presented as mean±standard error of the mean (SEM). Comparisons between groups were performed using a one-way between-groups analysis of variance with post-hoc comparisons using the Tukey HSD test. A value of p<0.05 was considered statistically significant.

### Results

Decreased CD39 on Pulmonary Endothelium and Plexiform Lesions from IPAH Patients

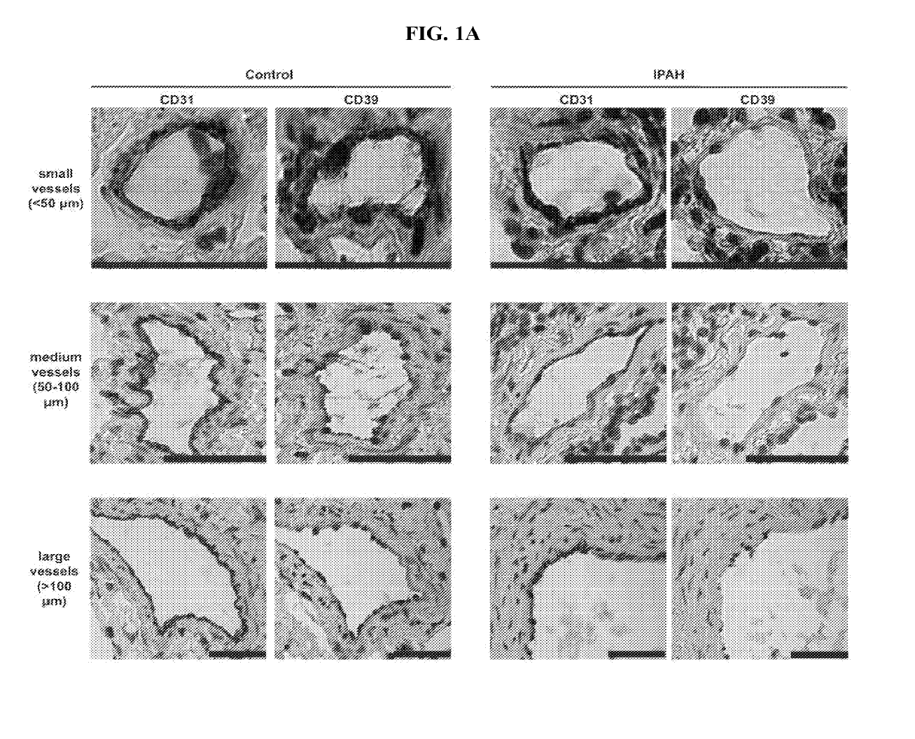

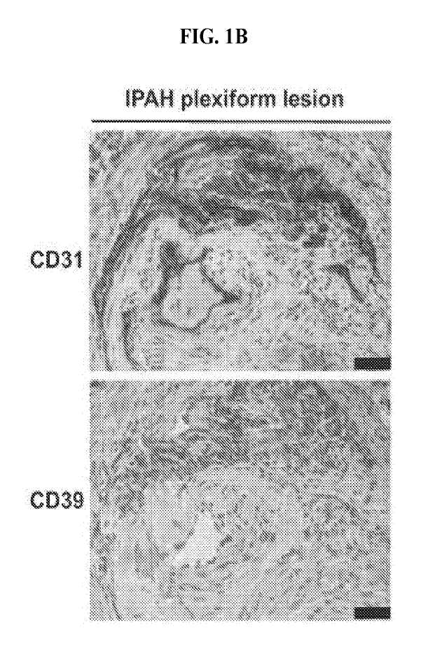

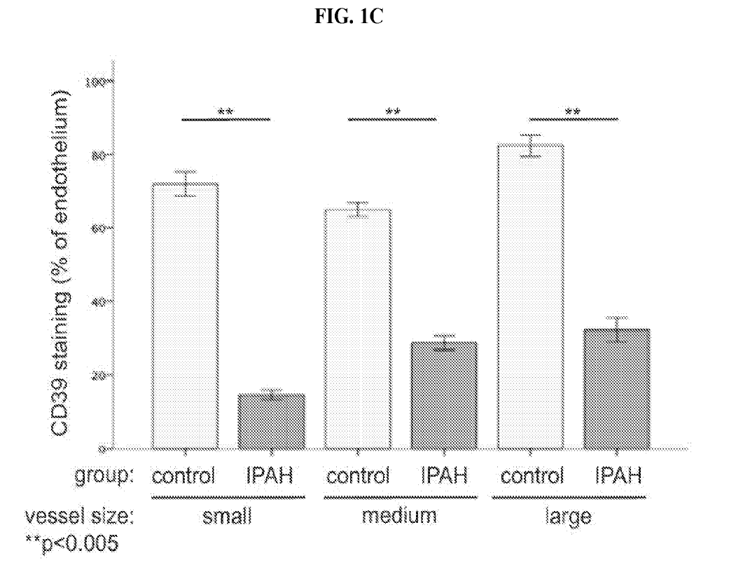

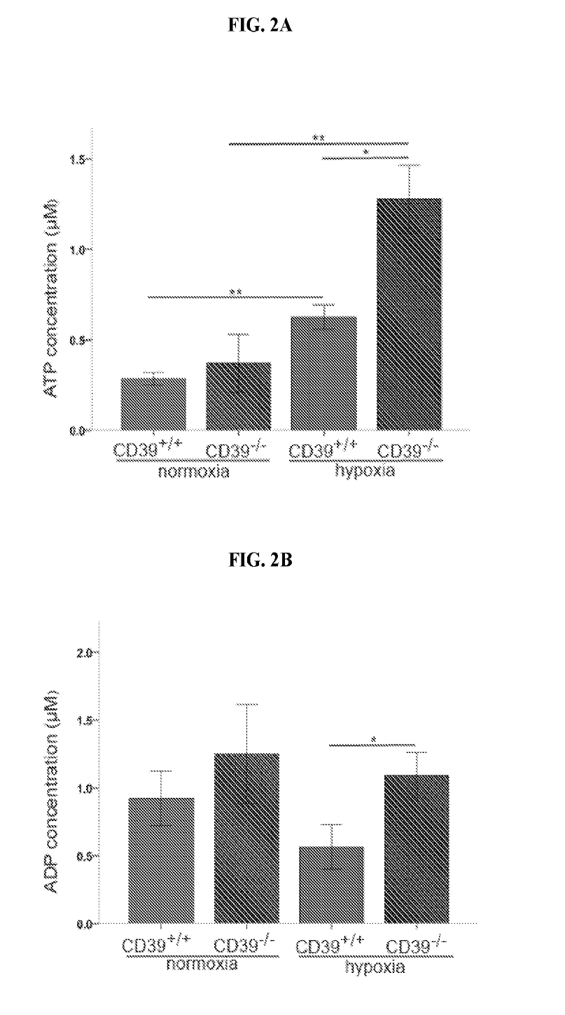

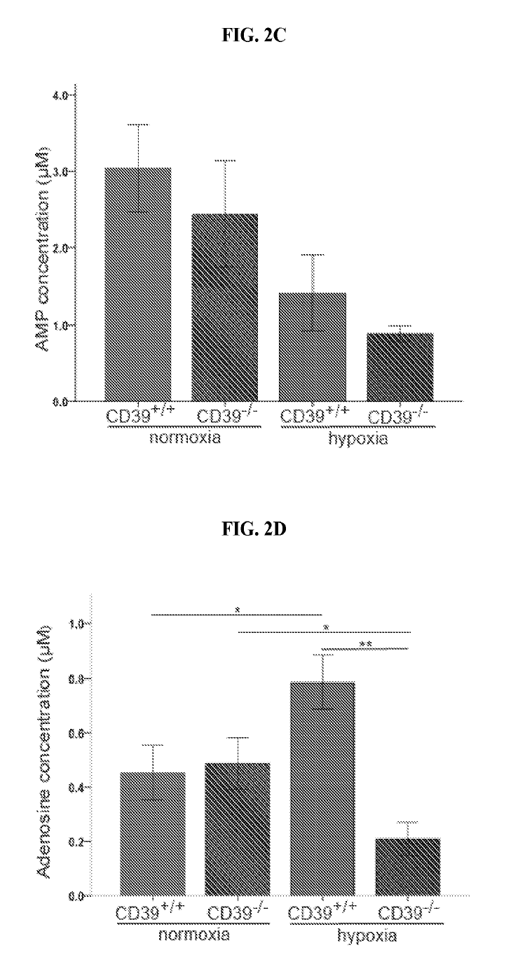

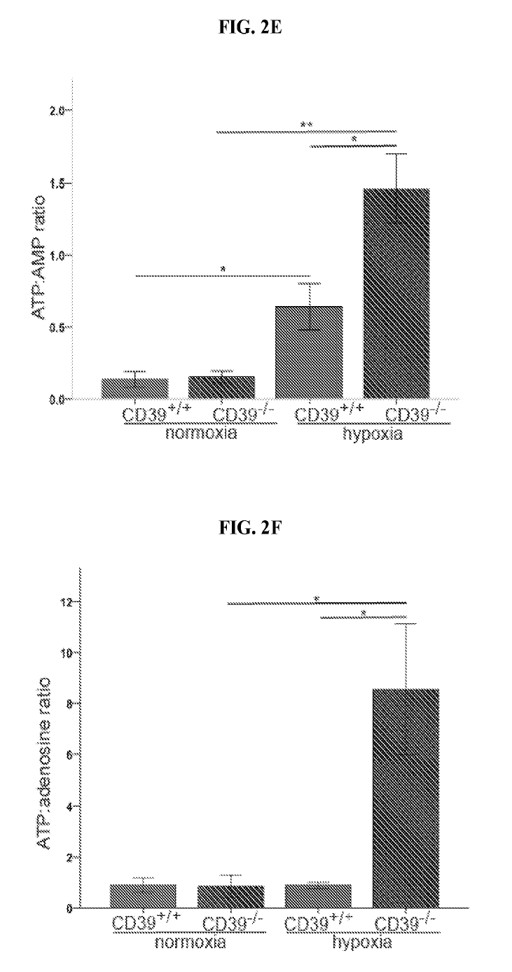

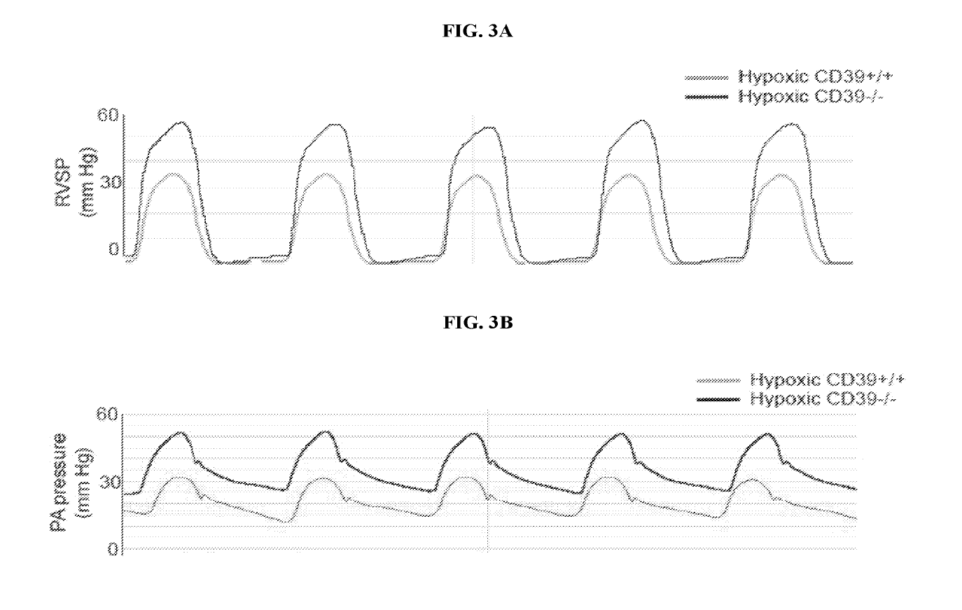

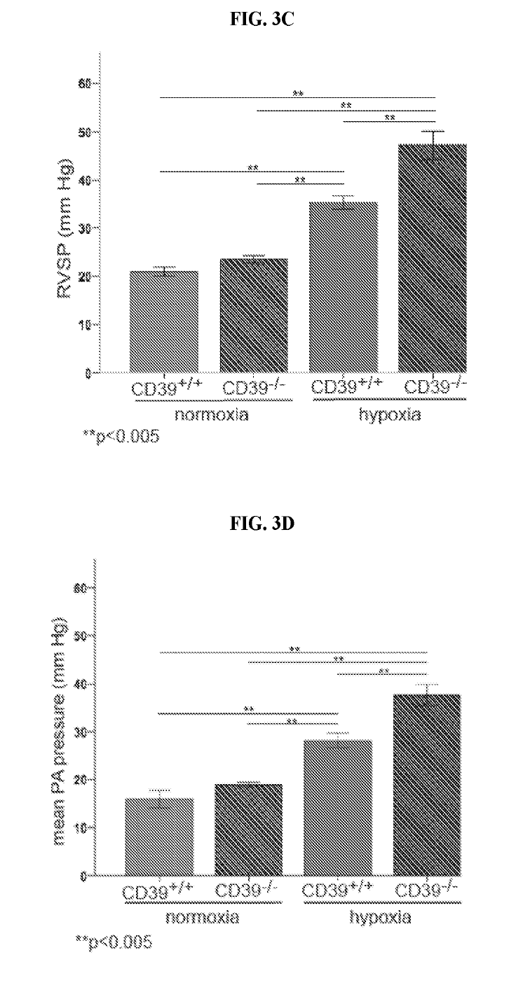

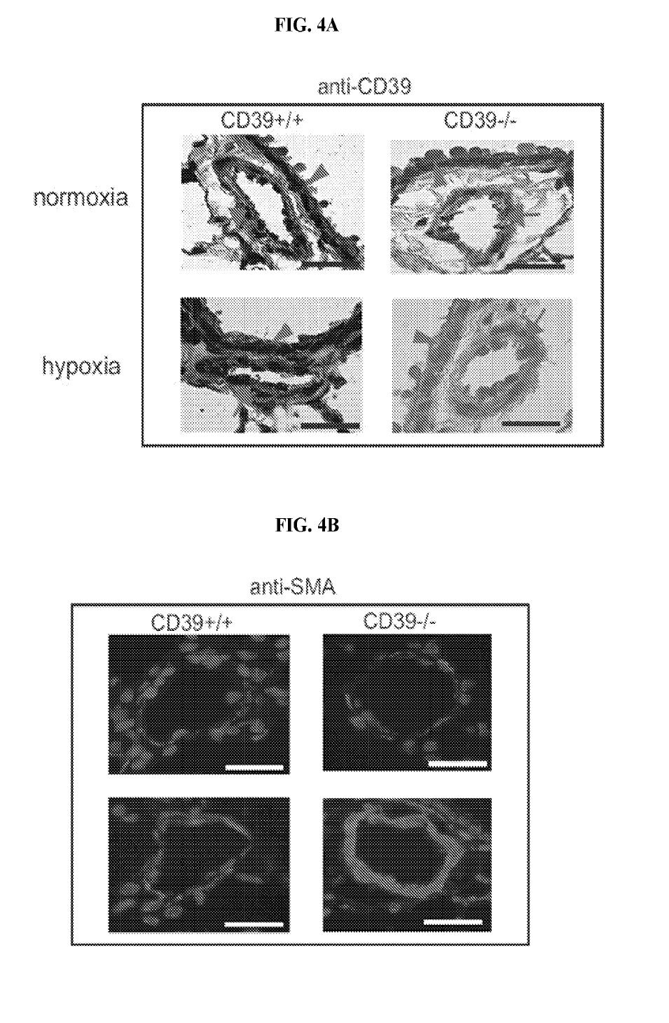

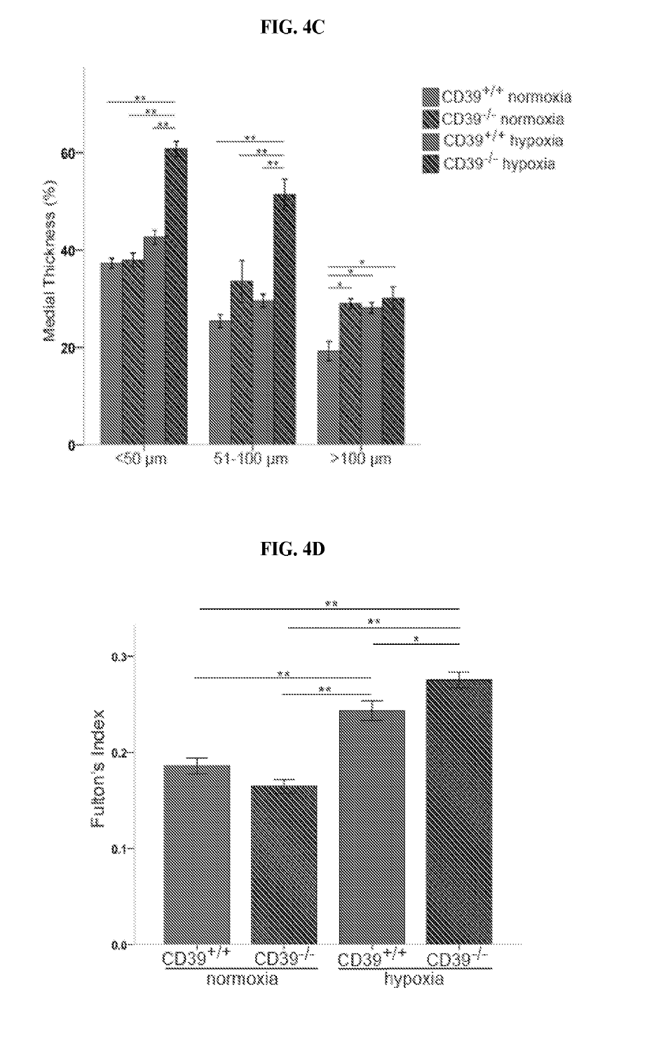

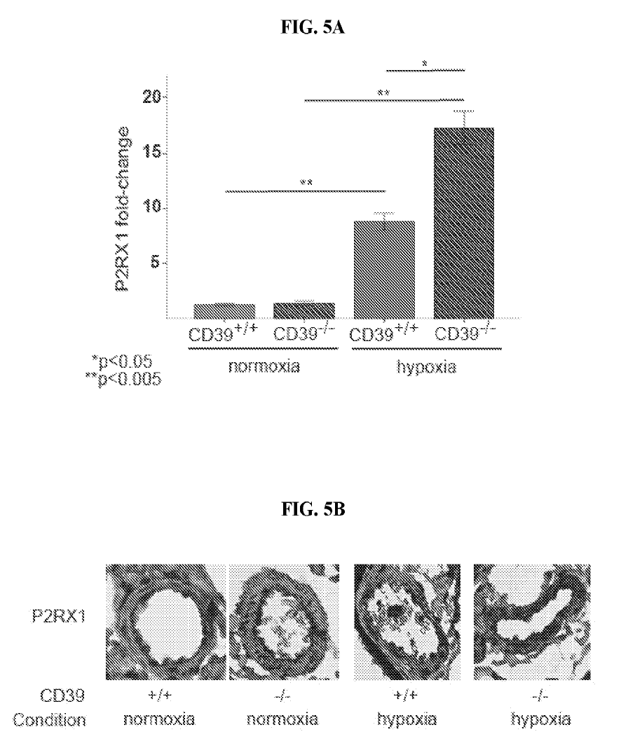

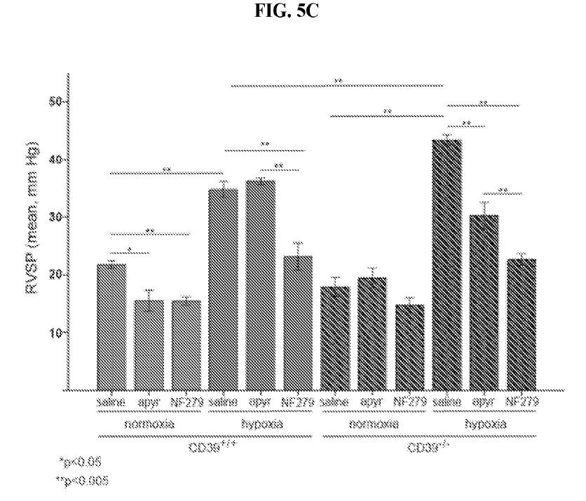

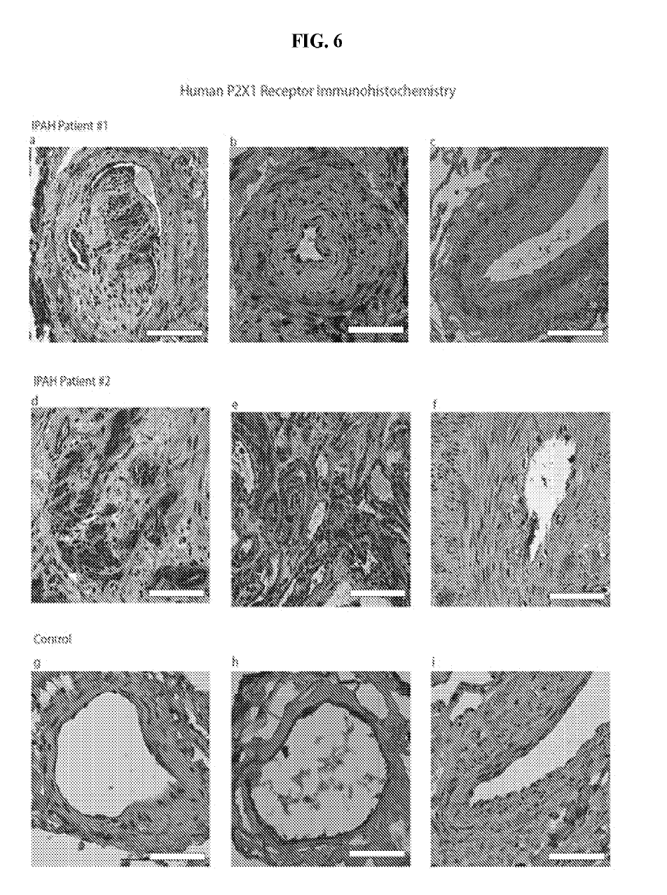

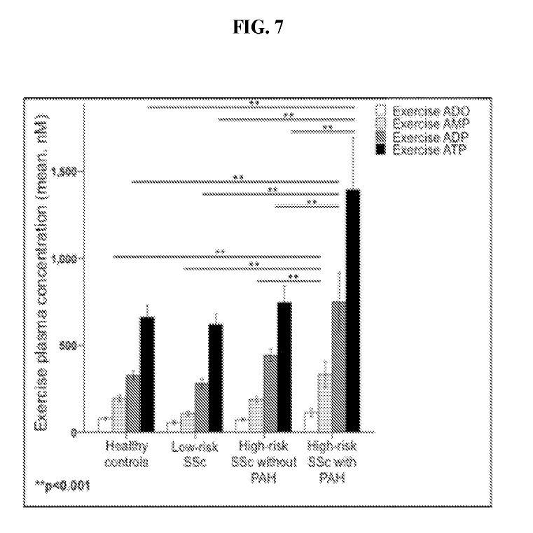

Experiments were conducted during development of embodiments herein to determine whether patients with IPAH have decreased CD39 on both the pulmonary vascular endothelium of all vessel sizes, as well as within the angiomatoid proliferative lesions (plexiform lesions) commonly found in patients with severe IPAH (ref 54; incorporated by reference in its entirety). Immunohistochemistry using anti-CD39 antibody confirmed the presence of CD39 on CD31-positive pulmonary endothelium in vessels of all sizes (<50 μm, 50-100 μm and >100 μm) from donors with healthy lungs (“controls” in FIG. 1a). However, lung tissue from patients with IPAH showed markedly decreased pulmonary endothelial CD39 (FIG. 1a). Typical plexiform lesions containing vascular channels lined by CD31-positive endothelial cells were identified in lung samples from IPAH patients (FIG. 1b). Notably, CD39 was not present within these endothelial-rich lesions. (FIG. 1b). Quantification of the mean area of CD39-positive staining for 10 small (<50 μm) vessels selected at random from the lungs of 2 IPAH and 2 control patients (FIG. 1c) shows a statistically significant decrease in CD39-positive endothelial staining in IPAH patients. In order to determine if a decrease in CD39 occurs in non-vascular lung structures we examined expression in the pulmonary airway epithelium (FIG. 1d). Notably, there was no decrease in pulmonary airway epithelial CD39 expression in IPAH patients compared to controls without airway disease.

### Global Deletion of Murine CD39

In order to further investigate the contribution of decreased CD39 to the pathobiology of PAH CD39-deficient mice we generated utilizing a ploxPFLpneo targeting vector. PCR of tail tips from wild-type (CD39^{+/+}) and CD39 knockout (CD39^{-/-}) mice confirmed the genotypes of each animal. qRT-PCR confirmed a lack of CD39 expression in multiple organs from CD39^{-/-} mice. Western blotting confirmed the absence of CD39 protein in the lungs of CD39^{-/-} mice. These experiments confirm deletion of CD39.

### CD39 Deletion Increases the Plasma ATP-to-Adenosine Ratio in Mice Exposed to Hypoxia

Experiments were conducted during development of embodiments herein to determine whether global deletion of CD39 would alter nucleotides and adenosine both in the circulation and in the local milieu, creating an ATP-rich and adenosine-poor intravascular environment. Using a liquid chromatography-mass spectrometry (LC-MS) platform, it was determined that hypoxic CD39^{+/+} and CD39^{-/-} mice both exhibited significantly increased plasma ATP concentrations, compared to normoxic controls (FIG. 2a). Among the hypoxic groups, the ATP concentration was significantly higher in CD39^{-/-} mice, compared to wild-type mice (FIG. 2a). Hypoxic CD39^{-/-} mice were also found to have higher ADP concentrations compared to hypoxic wild-type controls (FIG. 2b). Both CD39^{+/+} and CD39^{-/-} mice had a hypoxia-induced decrease in AMP concentrations, with a trend towards lower concentrations in both normoxic and hypoxic CD39^{-/-} mice, compared to wild-type controls (FIG. 2c). Normoxic wild-type and CD39^{-/-} mice had similar adenosine concentrations (FIG. 2d). In wild-type mice, hypoxia induced a significant increase in plasma adenosine. However, a significant decrease in adenosine concentration was noted in hypoxic CD39^{-/-} mice (FIG. 2d).

In order to assess the overall effect of CD39 gene deletion on a complex system that involves the sequential generation of products that become substrates for subsequent enzymatic reactions, two important ratios were compared. The ATP-to-AMP ratio compares the starting substrate (ATP) and final product (AMP) of CD39 ectonucleotidase activity. As shown in FIG. 2e, hypoxic CD39^{-/-} mice had a significantly elevated ratio compared to hypoxic wild-type, and both normoxic, controls. The ATP-to-adenosine ratio reflects the combined actions of CD39 and CD73, as the latter converts AMP to adenosine. Hypoxic CD39^{-/-} mice had strikingly higher plasma ATP-to-adenosine ratios compared to all other groups (FIG. 20. Of note, hypoxic CD39^{+/+} mice exhibited an increase in both plasma ATP and adenosine concentrations compared to their normoxic controls, which is consistent with prior studies (ref 5, 14; incorporated by reference in their entireties). However, hypoxic but not normoxic CD39^{-/-} mice exhibited a statistically significant increases in ATP and ADP compared to CD39^{+/+} controls.

### CD39 Deletion Results in Severe Pulmonary Arterial Hypertension in Mice Exposed to Hypoxia

Representative actual waveform tracings shown in FIGS. 3a and 3b illustrate the extreme differences in right ventricular systolic pressure (RVSP) and mean pulmonary artery (PA) pressure, respectively, in hypoxic CD39^{-/-} mice compared to CD39^{+/+} mice. Under normoxic conditions, there was no difference in mean RVSP (FIG. 3c) and mean PA pressure (FIG. 3d) between CD39^{+/+} and CD39^{-/-} mice. Following 4 weeks of hypoxia (10% oxygen), CD39^{-/-} mice developed a statistically significant increase in both pressure parameters compared to hypoxic CD39^{+/+}, normoxic CD39^{-/-}, and normoxic CD39^{+/+} mice. In order to assess the possible effects of differences in heart rate on intra-cardiac pressures, heart rates were obtained after administering isoflurane, but prior to the surgical procedure. There were no differences in heart rate between the groups (Table 1). Systemic blood pressures increased in response to hypoxia (Table 1), though there were no significant differences in pressure increases between the CD39^{+/+} and CD39^{-/-} mice. These findings confirm that the extreme increase in pulmonary arterial pressure in CD39^{-/-} mice compared to CD39^{+/+} mice was secondary to an increase in pulmonary vascular resistance, rather than an increase in heart rate or induction of disproportionate systemic hypertension. Arterial blood gas analysis (Table 1) showed a similar increase in hemoglobin and decrease in pO₂ in both CD39^{+/+} and CD39^{-/-} mice, confirming that the extreme pulmonary arterial hypertension in CD39^{-/-} mice was not secondary to disproportionate erythrocytosis or hypoxemia.

TABLE 1
Physiologic parameters for normoxic and hypoxic mice based upon genotype.

| | Normoxia | | | Hypoxia | | |
|---|---|---|---|---|---|---|
| | CD39^{+/+} | CD39^{-/-} | p | CD39^{+/+} | CD39^{-/-} | p |
| Heart rate | 444.8 ± 26.2 | 431.0 ± 31.3 | n.s. | 429.3 ± 18.5 | 472.0 ± 12.7 | n.s. |
| (bpm) | | | | | | |
| Systolic blood | 94.8 ± 3.6 | 96.1 ± 2.1 | n.s. | 111.6 ± 1.6 | 105.0 ± 4.0 | n.s. |
| pressure | | | | | | |
| (mmHg) | | | | | | |
| Diastolic | 65.8 ± 1.3 | 71.0 ± 4.8 | n.s. | 82.9 ± 1.0 | 82.3 ± 3.0 | n.s. |
| blood | | | | | | |
| pressure | | | | | | |
| (mmHg) | | | | | | |
| Mean arterial | 85.7 ± 1.8 | 86.4 ± 2.0 | n.s. | 102.3 ± 1.5 | 97.3 ± 3.6 | n.s. |
| pressure | | | | | | |
| (mmHg) | | | | | | |
| Hemoglobin | 12.6 ± 1.0 | 14.5 ± 1.0 | n.s. | 25.0 ± 0.7 | 26.1 ± 0.2 | n.s. |
| (g/dl) | | | | | | |
| pO₂ | 112.9 ± 4.8 | 104.4 ± 3.5 | n.s. | 52.1 ± 7.8 | 48.5 ± 8.2 | n.s. |
| (mmHg) | | | | | | |

Abbreviations:
bpm, beats per minute:
g/dl, grams per deciliter;
mmHg, millimeters of mercury;
PO₂, partial pressure of oxygen.
Values are means ± SEM

### CD39 Deletion Exacerbates Hypoxic Pulmonary Arterial and Right Ventricular Remodeling

The effect of hypoxia on CD39 is shown in FIG. 4a where representative CD39 immunostaining shows a similar pattern in CD39^{+/+} mice exposed to normoxia and hypoxia, and no staining in CD39^{-/-} mice. The effect of hypoxia on pulmonary arterial remodeling was determined by measuring the medial thickness of small, medium and large arteries. Representative photomicrographs of small pulmonary arteries from CD39^{+/+} and CD39^{-/-} mice exposed to normoxia and hypoxia are shown in FIG. 4b. Immunolabeling with anti-αSMA shows a significantly thicker medial layer in hypoxic CD39^{-/-} mice, compared to the other groups. Analysis of pulmonary arterial medial thickness in pulmonary arteries of various sizes confirmed that hypoxia induced significantly greater medial thickness in the small (<50 μm) and medium (51-100 μm) pulmonary arteries of CD39^{-/-} mice compared to hypoxic CD39^{+/+}, normoxic CD39^{-/-}, and normoxic CD39^{+/+} mice (FIG. 4c). It is important to note that normoxic CD39^{-/-} mice also had significantly increased medial thickness in comparison to normoxic CD39^{+/+} mice.

Hypoxia also induced greater right ventricular hypertrophy in CD39^{-/-} compared to CD39^{+/+} mice (FIG. 4d), as shown by a statistically significant increase in Fulton's Index (the ratio of RV weight to the sum of the left ventricular plus septal weights) of hypoxic CD39^{-/-} mice, compared to the other groups.

### CD39 Deletion Results in Altered Lung Purinergic Receptor Expression

Given that intravascular nucleotides are ligands for purinergic receptors that regulate vascular tone, the transcription levels of major receptors were profiled in whole lung homogenates from each of the mouse groups (Table 2, 3 and 4). Amongst the ligand-gated P2X purinoreceptors, hypoxia significantly up-regulated the P2X1 receptor in CD39^{+/+} (almost a 9-fold increase) and, to an even greater extent, CD39^{-/-} mice (a 17-fold increase), compared to normoxic controls (Table 2 and FIG. 5a). P2Y2 was the only G protein-coupled purinoreceptor significantly affected by hypoxia, and CD39^{-/-} mice exhibited a significant up-regulation of this receptor compared to both hypoxic CD39^{+/+} mice and normoxic controls (Table 3). The adenosine A2A was up-regulated by hypoxia in both CD39^{+/+} (a 2-fold increase) and CD39^{-/-} (a 6-fold increase) mice, compared to normoxic controls (Table 4).

TABLE 2

| | Average mRNA fold-change compared to | | | | |
|---|---|---|---|---|---|
| | CD39 +/+ normoxic mice | | | | |
| P2X receptor | P2X1 | P2X2 | P2X3 | P2X4 | P2X5 |
| CD39 +/+ normoxic | 1.00 | 1.00 | 1.00 | 1.00 | 1.00 |
| CD39 −/− normoxic | 1.36 | 1.18 | 1.10 | 1.03 | 1.01 |
| CD39 +/+ hypoxic | 8.73* | 1.02 | 1.09 | 0.86 | 1.32 |
| CD39 −/− hypoxic | 17.06* | 1.11 | 1.27 | 0.99 | 1.33 |

*p < 0.05 for CD39 −/− hypoxic vs. CD39 +/+ hypoxic

TABLE 3

| | Average mRNA fold-change compared to | | | | |
|---|---|---|---|---|---|
| | CD39 +/+ normoxic mice | | | | |
| P2Y receptor | P2Y1 | P2Y2 | P2Y4 | P2Y6 | P2Y12 |
| CD39 +/+ normoxic | 1.00 | 1.00 | 1.00 | 1.00 | 1.00 |
| CD39 −/− normoxic | 0.89 | 1.01 | 1.14 | 0.99 | 1.02 |
| CD39 +/+ hypoxic | 0.73 | 1.65* | 1.31 | 0.80 | 0.84 |
| CD39 −/− hypoxic | 1.59 | 3.34* | 1.96 | 0.89 | 1.86 |

*p < 0.05 for CD39 −/− hypoxic vs. CD39 +/+ hypoxic

TABLE 4

Average mRNA fold-change compared
to CD39 +/+ normoxic mice

| Adenosine receptor | A1 | A2A | A2B | A3 |
|---|---|---|---|---|
| CD39 +/+ normoxic | 1.00 | 1.00 | 1.00 | 1.00 |
| CD39 −/− normoxic | 1.20 | 0.91 | 0.77 | 0.69 |
| CD39 +/+ hypoxic | 1.11 | 2.16* | 0.93 | 0.37 |
| CD39 −/− hypoxic | 1.49 | 6.52* | 0.85 | 0.59 |

*p < 0.05 for CD39 −/− hypoxic vs. CD39 +/+ hypoxic

Reconstitution of Hypoxic CD39⁻ Mice with Soluble Apyrase Decreases Pulmonary Hypertension and Treatment with a P2X1 Receptor Antagonist Prevents Severe Pulmonary Hypertension

Based upon findings of increased intravascular ATP and ADP, significant up-regulation of the P2X1 receptor, and severe pulmonary hypertension in hypoxic CD39^{-/-} mice, two sets of rescue experiments were designed aimed at reversing the PH phenotype. The first rescue experiment involved the reconstitution of intravascular ectonucleotidase activity. Soluble potato tuber apyrase has known ATPase and ADPase activity, and has been used in prior studies involving CD39^{-/-} mice (ref 24, 44; incorporated by reference in their entireties). Sequence homology of this apyrase is similar to human and murine CD39 (ref 19; incorporated by reference in its entirety). As shown in FIG. 5c, continuous subcutaneous administration of soluble apyrase during four weeks of hypoxia significantly decreased the RVSP in normoxic CD39^{+/+} and hypoxic CD39^{-/-} mice, compared to control mice that received continuous saline infusions. Of note, apyrase did not decrease the RVSP in hypoxic CD39^{-/-} mice to levels found in normoxic CD39^{+/+} mice, indicating that the conversion of ATP and ADP to downstream products such as AMP and adenosine alone was not sufficient to completely reverse the PH phenotype. The second rescue experiment involved the continuous infusion of NF279 (8,8′-[Carbonylbis(imino-4,1-phenylenecarbonylimino-4,1-phenylenecarbonylimino)]bis-1,3,5-naphthalenetrisulfonic acid hexasodium salt):

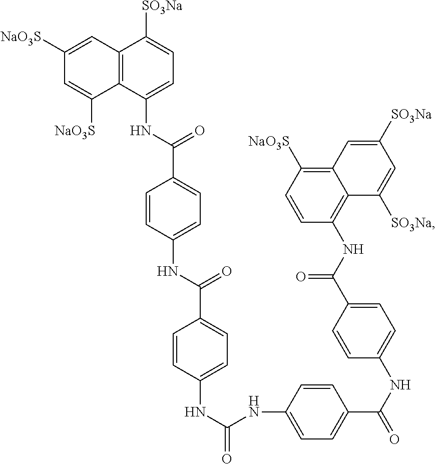

a sensitive and specific P2X1 antagonist, during four weeks of hypoxia. As shown in FIG. 5c, NF279 prevented the development of PH in all groups, including hypoxic CD39^{-/-} mice.
Pulmonary Vessels from Patients with IPAH have Increased P2X1 Receptors

The mouse experiments described herein implicated increased pulmonary vascular P2X1 receptors in the pathobiology of experimental PH. In order to determine the significance of this finding in humans, immunohistochemical staining was performed for the P2X1 receptor in human lung sections. As shown in FIG. 6, pulmonary vessels from 2 patients with IPAH exhibit increased P2X1 receptor staining in plexiform lesions, remodeled vessels with medial hypertrophy, and in larger vessels compared to individuals without lung disease.

### Example 2

Experiments conducted during development of embodiments herein in a CD39-deleted mouse (See, e.g., Example 1) have demonstrated that alterations in purinergic signaling result in severe pulmonary arterial hypertension (PAH). Described below are experiments conducted during development of embodiments herein to demonstrate that human individuals with scleroderma-associated PAH have a plasma nucleotide concentration profiles during rest and/or exercise that is distinct from patients without PAH.

### Methods

Informed consent was obtained from individuals in four groups: (1) “Healthy controls” (n=37); (2) “Low-risk scleroderma” patients without risk factors for PAH (n=23); (3) “High risk scleroderma” patients with PAH risk factors but found not to have PAH by right heart catheterization (RHC, n=31); and (4) High risk scleroderma” patients with risk factors for PAH and found to have PAH by RHC (n=17). All individuals participated in a symptom-limited exercise protocol using a supine ergometer either in the catheterization laboratory (high risk individuals) or a clinical research unit (healthy controls and low risk individuals). Prior to exercise and again at peak exercise, blood was drawn from the antecubital vein into syringes pre-filled with a stop solution formulated to minimize the metabolism of nucleotides and adenosine. ATP, ADP, AMP and ADO concentrations were determined using liquid chromatography-mass spectrometry. Relationships between nucleotide concentrations and the four groups were investigated using one-way ANOVA and the Pearson product-moment correlation coefficient.

### Results

No significant differences in resting nucleotide and ADO concentrations were found between groups. The high-risk scleroderma with PAH group had statistically significant increases in mean peak exercise ATP, ADP and AMP concentrations compared to each of the other groups (FIG. 7). The effect size was large (eta squared of 0.26). There were large, positive correlations between the presence of PAH and ATP (r=0.50, p<0.0001) and ADP (r=0.53, p<0.0001) and a medium correlation with AMP (r=0.39, p<0.0001) concentrations at peak exercise. These data demonstrate that in patients with scleroderma, increased ATP, ADP and AMP concentrations measured during exercise are associated with the presence of PAH. In some embodiments, these biomarkers find use in the detection (e.g., early detection) of scleroderma-associated PAH. These data indicate that agents that degrade purinergic signaling ligands in patients with scleroderma is useful in the treatment and/or prevention of PAH.

All publications and patents listed below and/or provided herein are incorporated by reference in their entireties. Various modifications and variations of the described compositions and methods of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the present invention.

### REFERENCES

The following references, some of which are referenced above by number (e.g., ref X) are herein incorporated by reference in their entireties.
1. Alencar A K, Pereira S L, Montagnoli T L, Maia R C, Kummerle A E, Landgraf S S, Caruso-Neves C, Ferraz E B, Tesch R, Nascimento J H, de Sant'Anna C M, Fraga C A, Barreiro E J, Sudo R T, and Zapata-Sudo G. Beneficial effects of a novel agonist of the adenosine A2A receptor on monocrotaline-induced pulmonary hypertension in rats. Br J Pharmacol 169: 953-962, 2013.
2. Beppu H, Ichinose F, Kawai N, Jones R C, Yu P B, Zapol W M, Miyazono K, Li E, and Bloch K D. BMPR-II heterozygous mice have mild pulmonary hypertension and an impaired pulmonary vascular remodeling response to prolonged hypoxia. American journal of physiology Lung cellular and molecular physiology 287: L1241-1247, 2004.
3. Bergfeld G R, and Forrester T. Release of ATP from human erythrocytes in response to a brief period of hypoxia and hypercapnia. Cardiovasc Res 26: 40-47, 1992.
4. Bodin P, and Burnstock G. Synergistic effect of acute hypoxia on flow-induced release of ATP from cultured endothelial cells. Experientia 51: 256-259, 1995.
5. Burnstock G. Dual control of local blood flow by purines. Ann N Y Acad Sci 603: 31-44; discussion 44-35, 1990.
6. Burnstock G. Purinergic nerves. Pharmacol Rev 24: 509-581, 1972.
7. Burnstock G. Purinergic regulation of vascular tone and remodelling. Auton Autacoid Pharmacol 29: 63-72, 2009.
8. Burnstock G, and Ralevic V. New insights into the local regulation of blood flow by perivascular nerves and endothelium. Br J Plast Surg 47: 527-543, 1994.
9. Ciuclan L, Bonneau O, Hussey M, Duggan N, Holmes A M, Good R, Stringer R, Jones P, Morrell N W, Jarai G, Walker C, Westwick J, and Thomas M. A novel murine model of severe pulmonary arterial hypertension. American journal of respiratory and critical care medicine 184: 1171-1182, 2011.
10. Daley E, Emson C, Guignabert C, de Waal Malefyt R, Louten J, Kurup V P, Hogaboam C, Taraseviciene-Stewart L, Voelkel N F, Rabinovitch M, Grunig E, and Grunig G. Pulmonary arterial remodeling induced by a Th2 immune response. J Exp Med 205: 361-372, 2008.
11. Damer S, Niebel B, Czeche S, Nickel P, Ardanuy U, Schmalzing G, Rettinger J, Mutschler E, and Lambrecht G. NF279: a novel potent and selective antagonist of P2X receptor-mediated responses. Eur J Pharmacol 350: R5-6, 1998.
12. Das M, Fessel J, Tang H, and West J. A process-based review of mouse models of pulmonary hypertension. Pulmonary circulation 2: 415-433, 2012.
13. Dawicki D D, Chatterjee D, Wyche J, and Rounds S. Extracellular ATP and adenosine cause apoptosis of pulmonary artery endothelial cells. Am J Physiol 273: L485-494, 1997.
14. Eltzschig H K, Abdulla P, Hoffman E, Hamilton K E, Daniels D, Schonfeld C, Loftier M, Reyes G, Duszenko M, Karhausen J, Robinson A, Westerman K A, Coe I R, and Colgan S P. HIF-1-dependent repression of equilibrative nucleoside transporter (ENT) in hypoxia. J Exp Med 202: 1493-1505, 2005.
15. Eltzschig H K, Kohler D, Eckle T, Kong T, Robson S C, and Colgan S P. Central role of Sp1-regulated CD39 in hypoxia/ischemia protection. Blood 113: 224-232, 2009.
16. Erlinge D, and Burnstock G. P2 receptors in cardiovascular regulation and disease. Purinergic Signal 4: 1-20, 2008.
17. Gomez-Arroyo J, Saleem S J, Mizuno S, Syed A A, Bogaard H J, Abbate A, Taraseviciene-Stewart L, Sung Y, Kraskauskas D, Farkas D, Conrad D H, Nicolls M R, and Voelkel N F. A brief overview of mouse models of pulmonary arterial hypertension: problems and prospects. American journal of physiology Lung cellular and molecular physiology 302: L977-991, 2012.
18. Gorman M W, Feigl E O, and Buffington C W. Human plasma ATP concentration. Clin Chem 53: 318-325, 2007.
19. Handa M, and Guidotti G. Purification and cloning of a soluble ATP-diphosphohydrolase (apyrase) from potato tubers (Solanum tuberosum). Biochemical and biophysical research communications 218: 916-923, 1996.
20. Hara Y, Sassi Y, Guibert C, Gambaryan N, Dorfmuller P, Eddahibi S, Lompre A M, Humbert M, and Hulot J S. Inhibition of MRP4 prevents and reverses pulmonary hypertension in mice. J Clin Invest 121: 2888-2897, 2011.
21. Hart M L, Gorzolla I C, Schittenhelm J, Robson S C, and Eltzschig H K. SP1-dependent induction of CD39 facilitates hepatic ischemic preconditioning. Journal of immunology 184: 4017-4024, 2010.
22. Haynes J, Jr., Obiako B, Thompson W J, and Downey J. Adenosine-induced vasodilation: receptor characterization in pulmonary circulation. Am J Physiol 268: H1862-1868, 1995.
23. Helenius M H, Vattulainen S, Orcholski M, Aho J, Komulainen A, Taimen P, Wang L, de Jesus Perez V A, Koskenvuo J W, and Alastalo T P. Suppression of endothelial CD39/ENTPD1 is associated with pulmonary vascular remodeling in pulmonary arterial hypertension. American journal of physiology Lung cellular and molecular physiology 308: L1046-1057, 2015.
24. Hyman M C, Petrovic-Djergovic D, Visovatti S H, Liao H, Yanamadala S, Bouis D, Su E J, Lawrence D A, Broekman M J, Marcus A J, and Pinsky D J. Self-regulation of inflammatory cell trafficking in mice by the leukocyte surface apyrase CD39. J Clin Invest 119: 1136-1149, 2009.
25. Inscho E W, Cook A K, Imig J D, Vial C, and Evans R J. Physiological role for P2X1 receptors in renal microvascular autoregulatory behavior. J Clin Invest 112: 1895-1905, 2003.
26. Kaczmarek E, Koziak K, Sevigny J, Siegel J B, Anrather J, Beaudoin A R, Bach F H, and Robson S C. Identification and characterization of CD39/vascular ATP diphosphohydrolase. J Biol Chem 271: 33116-33122, 1996.
27. Katsuragi T, Tokunaga T, Ogawa S, Soejima O, Sato C, and Furukawa T. Existence of ATP-evoked ATP release system in smooth muscles. J Pharmacol Exp Ther 259: 513-518, 1991.
28. Kylhammar D, Bune L T, and Radegran G. P2Y and P2Y receptors in hypoxia- and adenosine diphosphate-induced pulmonary vasoconstriction in vivo in the pig. Eur J Appl Physiol 2014.
29. Lew M J, and White T D. Release of endogenous ATP during sympathetic nerve stimulation. Br J Pharmacol 92: 349-355, 1987.
30. Liu Y, Zhang L, Wang C, Roy S, and Shen J. Purinergic P2Y2 Receptor Control of Tissue Factor Transcription in Human Coronary Artery Endothelial Cells: NEW AP-1 TRANSCRIPTION FACTOR SITE AND NEGATIVE REGULATOR. J Biol Chem 291: 1553-1563, 2016.
31. Marcus A J, and Safier L B. Thromboregulation: multicellular modulation of platelet reactivity in hemostasis and thrombosis. FASEB journal: official publication of the Federation of American Societies for Experimental Biology 7: 516-522, 1993.
32. McAuslan B R, Reilly W G, Hannan G N, and Gole G A. Angiogenic factors and their assay: activity of formyl methionyl leucyl phenylalanine, adenosine diphosphate, heparin, copper, and bovine endothelium stimulating factor. Microvasc Res 26: 323-338, 1983.
33. McLaughlin V V, Archer S L, Badesch D B, Barst R J, Farber H W, Lindner J R, Mathier M A, McGoon M D, Park M H, Rosenson R S, Rubin L J, Tapson V F, Varga J, American College of Cardiology Foundation Task Force on Expert Consensus D, American Heart A, American College of Chest P, American Thoracic Society I, and Pulmonary Hypertension A. ACCF/AHA 2009 expert consensus document on pulmonary hypertension a report of the American College of Cardiology Foundation Task Force on Expert Consensus Documents and the American Heart Association developed in collaboration with the American College of Chest Physicians; American Thoracic Society, Inc.; and the Pulmonary Hypertension Association. Journal of the American College of Cardiology 53: 1573-1619, 2009.
34. McMillan M R, Burnstock G, and Haworth S G. Vasoconstriction of intrapulmonary arteries to P2-receptor nucleotides in normal and pulmonary hypertensive newborn piglets. Br J Pharmacol 128: 549-555, 1999.
35. Morgan J M, McCormack D G, Griffiths M J, Morgan C J, Barnes P J, and Evans T W. Adenosine as a vasodilator in primary pulmonary hypertension. Circulation 84: 1145-1149, 1991.
36. Morimatsu Y, Sakashita N, Komohara Y, Ohnishi K, Masuda H, Dahan D, Takeya M, Guibert C, and Marthan R. Development and characterization of an animal model of severe pulmonary arterial hypertension. J Vasc Res 49: 33-42, 2012.
37. Mulryan K, Gitterman D P, Lewis C J, Vial C, Leckie B J, Cobb A L, Brown J E, Conley E C, Buell G, Pritchard C A, and Evans R J. Reduced vas deferens contraction and male infertility in mice lacking P2X1 receptors. Nature 403: 86-89, 2000.
38. Nootens M, Schrader B, Kaufmann E, Vestal R, Long W, and Rich S. Comparative acute effects of adenosine and prostacyclin in primary pulmonary hypertension. Chest 107: 54-57, 1995.
39. Nori S, Fumagalli L, Bo X, Bogdanov Y, and Burnstock G. Coexpression of mRNAs for P2X1, P2X2 and P2X4 receptors in rat vascular smooth muscle: an in situ hybridization and R T-PCR study. J Vasc Res 35: 179-185, 1998.
40. Pearl R G. Adenosine produces pulmonary vasodilation in the perfused rabbit lung via an adenosine A2 receptor. Anesth Analg 79: 46-51, 1994.
41. Pinsky D J, Broekman M J, Peschon J J, Stocking K L, Fujita T, Ramasamy R, Connolly E S, Jr., Huang J, Kiss S, Zhang Y, Choudhri T F, McTaggart R A, Liao H, Drosopoulos JI-1, Price V L, Marcus A J, and Maliszewski C R. Elucidation of the thromboregulatory role of CD39/ectoapyrase in the ischemic brain. J Clin Invest 109: 1031-1040, 2002.
42. Pluskota E, Ma Y, Bledzka K M, Bialkowska K, Soloviev D A, Szpak D, Podrez E A, Fox P L, Hazen S L, Dowling J J, Ma Y Q, and Plow E F. Kindlin-2 regulates hemostasis by controlling endothelial cell-surface expression of ADP/AMP catabolic enzymes via a clathrin-dependent mechanism. Blood 122: 2491-2499, 2013.
43. Rettinger J, Schmalzing G, Darner S, Muller G, Nickel P, and Lambrecht G. The suramin analogue NF279 is a novel and potent antagonist selective for the P2X(1) receptor. Neuropharmacology 39: 2044-2053, 2000.
44. Reutershan J, Vollmer I, Stark S, Wagner R, Ngamsri K C, and Eltzschig H K. Adenosine and inflammation: CD39 and CD73 are critical mediators in LPS-induced PMN trafficking into the lungs. FASEB journal: official publication of the Federation of American Societies for Experimental Biology 23: 473-482, 2009.
45. Robson S C, Sevigny J, and Zimmermann H. The E-NTPDase family of ectonucleotidases: Structure function relationships and pathophysiological significance. Purinergic Signal 2: 409-430, 2006.
46. Ryan J, Bloch K, and Archer S L. Rodent models of pulmonary hypertension: harmonisation with the world health organisation's categorisation of human P H. Int j Clin Pract Suppl 15-34, 2011.
47. Saadjian A Y, Paganelli F, Gaubert M L, Levy S, and Guieu R P. Adenosine plasma concentration in pulmonary hypertension. Cardiovasc Res 43: 228-236, 1999.
48. Schindelin J, Arganda-Carreras I, Frise E, Kaynig V, Longair M, Pietzsch T, Preibisch S, Rueden C, Saalfeld S, Schmid B, Tinevez J Y, White D J, Hartenstein V, Eliceiri K, Tomancak P, and Cardona A. Fiji: an open-source platform for biological-image analysis. Nat Methods 9: 676-682, 2012.
49. Silver P J, Walus K, and DiSalvo J. Adenosine-mediated relaxation and activation of cyclic AMP-dependent protein kinase in coronary arterial smooth muscle. J Pharmacol Exp Ther 228: 342-347, 1984.
50. Sitbon O, and Morrell N. Pathways in pulmonary arterial hypertension: the future is here. Eur Respir Rev 21: 321-327, 2012.
51. Stenmark K R, Meyrick B, Galie N, Mooi W J, and McMurtry I F. Animal models of pulmonary arterial hypertension: the hope for etiological discovery and pharmacological cure. American journal of physiology Lung cellular and molecular physiology 297: L1013-1032, 2009.
52. Taraseviciene-Stewart L, Kasahara Y, Alger L, Hirth P, Mc Mahon G, Waltenberger J, Voelkel N F, and Tuder R M. Inhibition of the VEGF receptor 2 combined with chronic hypoxia causes cell death-dependent pulmonary endothelial cell proliferation and severe pulmonary hypertension. FASEB journal: official publication of the Federation of American Societies for Experimental Biology 15: 427-438, 2001.
53. Thenappan T, Shah S J, Rich S, and Gomberg-Maitland M. A USA-based registry for pulmonary arterial hypertension: 1982-2006. The European respiratory journal 30: 1103-1110, 2007.
54. Tuder R M, Marecki J C, Richter A, Fijalkowska I, and Flores S. Pathology of pulmonary hypertension. Clin Chest Med 28: 23-42, vii, 2007.
55. Vial C, and Evans R J. P2X receptor expression in mouse urinary bladder and the requirement of P2X(1) receptors for functional P2X receptor responses in the mouse urinary bladder smooth muscle. Br J Pharmacol 131: 1489-1495, 2000.
56. Visovatti S H, Hyman M C, Bouis D, Neubig R, McLaughlin V V, and Pinsky D J. Increased CD39 nucleotidase activity on microparticles from patients with idiopathic pulmonary arterial hypertension. PloS one 7: e40829, 2012.
57. Xu M H, Gong Y S, Su M S, Dai Z Y, Dai S S, Bao S Z, Li N, Zheng R Y, He J C, Chen J F, and Wang X T. Absence of the adenosine A2A receptor confers pulmonary arterial hypertension and increased pulmonary vascular remodeling in mice. J Vasc Res 48: 171-183, 2011.
58. Yegutkin G G. Nucleotide- and nucleoside-converting ectoenzymes: Important modulators of purinergic signalling cascade. Biochimica et biophysica acta 1783: 673-694, 2008.

1. A method of treating pulmonary arterial hypertension (PAH) in a subject, comprising administering to the subject an agent that degrades extraellular ATP.

2. The method of claim 1, wherein the agent dephosphorylates ATP to form ADP, AMP, and/or adenosine.

3. The method of claim 1, wherein the agent is an enzyme.

4. The method of claim 3, wherein the enzyme is selected from the group consisting of an ectonucleotidase, an ectonucleotide pyrophosphatase/phosphodiesterase, and an alkaline phosphatase.

5. The method of claim 4, wherein the enzyme is ectonucleoside triphosphate diphosphohydrolase-1 (CD39) or an active variant or fragment thereof.

6. The method of claim 3, wherein administering comprises administering a polypeptide comprising the enzyme to the subject.

7. The method of claim 3, wherein administering comprises administering a polynucleotide encoding the enzyme to the subject, under conditions such that the enzyme is expressed within cells of the subject and then released into the extracellular environment.

8. A method of treating pulmonary arterial hypertension (PAH) in a subject, comprising administering to the subject an adenosine analogue.

9. The method of claim 8, wherein the adenosine analogue is an adenosine receptor agonist.

10. The method of claim 9, wherein the adenosine analogue is an A2A receptor agonist.

11. The method of claim 10, wherein the A2A receptor agonist is selected from the group consisting of: ATL-146e, YT-146 (2-(1-octynyl)adenosine), CGS-21680, DPMA (N6-(2-(3,5-dimethoxyphenyl)-2-(2-methylphenyl)ethyl)adenosine), Regadenoson, UK-432,097, Limonene, and NECA (5′-(N-Ethylcarboxamido)adenosine).

12. The method of claim 9, wherein the adenosine analogue is an A2B receptor agonist.

13. The method of claim 12, wherein the A2B receptor agonist is selected from the group consisting of: BAY 60-6583, NECA (N-ethylcarboxamidoadenosine), (S)-PHPNECA, LUF-5835, and LUF-5845.

14. The method of claim 9, wherein the adenosine analogue is an A3 receptor agonist.

15. The method of claim 14, wherein the A3 receptor agonist is selected from the group consisting of: 2-(1-Hexynyl)-N-methyladenosine, CF-101 (IB-MECA), CF-102, 2-Cl-IB-MECA, CP-532,903, Inosine, LUF-6000, and MRS-3558.

16. The method of claim 8, wherein the adenosine analogue is stable in an extracellular physiological environment.

17. A method of treating pulmonary arterial hypertension (PAH) in a subject, comprising administering to the subject purinergic receptor antagonist.

18. The method of claim 17, wherein the purinergic receptor is selected from the group consisting of P2X1, P2X2, P2X3, P2X4, P2X5, P2Y1, P2Y2, P2Y4, P2Y6, P2Y12, ADORA1, ADORA2A, ADORA2B, and ADORA3.

19. The method of claim 18, comprising administering a P2X1 antagonist.

20. The method of claim 19, wherein the P2X1 antagonist is selected from the list consisting of: NF449, NF279, TNP-ATP triethylammonium salt, Suramin, PPADS (pyridoxalphosphate-6-azophenyl-2′,4′-disulfonic acid), NF 023, Adenosine 2′,5′-diphosphate, PPNDS (Pyridoxal-5′-phosphate-6-(2′-naphthyl azo-6′-nitro-4′,8′-disulfonate)), Ro 0437626, and MRS 2159.

21. The method of claim 17, wherein the purinergic receptor is a small molecule.

22. The method of claim 17, wherein the purinergic receptor is an antibody or antibody fragment.

23. A method of treating pulmonary arterial hypertension (PAH) in a subject, comprising administering to the subject an inhibitor of ATP synthesis and/or ATP-release from cells into the extracellular environment.

24. The method of one of claims 1-23, wherein the agent is administered to the subject systemically.

25. The method of one of claims 1-23, wherein the agent is administered to the subject locally to the pulmonary system or vascular system.

26. The method of one of claims 1-23, wherein the agent is administered to the subject locally to a pulmonary artery.

27. The method of one of claims 1-23, further comprising co-administration with a treatment for PAH and/or PAH symptoms selected from the group consisting of: diuretics, digoxins, blood thinners, calcium channel blockers, vasoactive substances, prostaglandins/prostanoids, endothelin receptor antagonists, phosphodiesterase type 5 inhibitors, nitric oxide pathway antagonists, and activators of soluble guanylate cyclase.

28. The method of one of claims 1-23, further comprising co-administration with a therapeutic selected from the group consisting of: an anticoagulant, an antithrombotic agent, an antiplatelet agent, an anti-inflammatory agent, an anti-proliferative/immunosuppressive agent, a cytostatic drug, an antioxidant.
